(19) United States
(12) Patent Application Publication
(10) Pub. No.: US 2013/0023882 A1
(43) Pub. Date: Jan. 24, 2013

(54) DISCECTOMY DEVICES AND RELATED METHODS

(71) Applicants: Myra I. L. Fabro, San Jose, CA (US); Gary D. Zaretzka, Castro Valley, CA (US); Stewart Kume, Emerald Hills, CA (US); John Davis, Sunnyvale, CA (US); Sam Boong Park, Saratoga, CA (US)

(72) Inventors: Myra I. L. Fabro, San Jose, CA (US); Gary D. Zaretzka, Castro Valley, CA (US); Stewart Kume, Emerald Hills, CA (US); John Davis, Sunnyvale, CA (US); Sam Boong Park, Saratoga, CA (US)

(21) Appl. No.: 13/396,438

(22) Filed: Feb. 14, 2012

Related U.S. Application Data
(60) Provisional application No. 61/443,229, filed on Feb. 15, 2011.

(51) Int. Cl. A61B 17/16

(52) U.S. Cl. 606/80

(57) ABSTRACT

A tissue removal device may comprise a handheld housing, a motor, and a tissue removal mechanism coupled to the handheld housing. The tissue removal mechanism may comprise a tissue collection chamber coupled to a distal portion of the handheld housing, a jaw member comprising first and second jaw portions, where the first jaw portion is coupled to a first elongated member and the second jaw portion is coupled to a second elongated member configured to actuate the second jaw portion, a rotatable shaft disposed within a lumen of the first elongated member, a helical member disposed around at least a portion of the rotatable shaft, and an impeller coupled to at least one of a distal end of the helical member and a distal end of the rotatable shaft. Rotation of the rotatable shaft may effect rotation of the helical member and the impeller.

This application claims priority to U.S. Provisional Application Ser. No. 61/443,229, entitled “Discectomy Devices and Related Methods,” filed Feb. 15, 2011, which application is incorporated herein by reference in its entirety.

### BACKGROUND

Vertebral disc herniation is a common disorder where a portion of a vertebral disc, a cushion-like structure located between the vertebral bodies of the spine, bulges out or extrudes beyond the usual margins of the disc and the spine. Disc herniation is believed to be the result of excessive loading on the disc in combination with weakening of the annulus due to such factors as aging and genetics. Disc herniation and other degenerative disc diseases are also associated with spinal stenosis, a narrowing of the bony and ligamentous structures of the spine. Although disc herniation can occur anywhere along the perimeter of the disc, it occurs more frequently in the posterior and posterior-lateral regions of the disc, where the spinal cord and spinal nerve roots reside. Compression of these neural structures can lead to pain, parasthesias, weakness, urine and fecal incontinence and other neurological symptoms that can substantially impact basic daily activities and quality of life.

Temporary relief of the pain associated with disc herniation is often sought through conservative therapy, which includes positional therapy (e.g. sitting or bending forward to reduce pressure on the spine), physical therapy, and drug therapy to reduce pain and inflammation. When conservative therapy fails to resolve a patient's symptoms, surgery may be considered to treat the structural source of the symptoms. Surgical treatments for disc herniation traditionally involve open procedures that involve dissection of muscle, connective tissue and bone along a patient's back as well as nerve manipulations to achieve adequate surgical exposure. For example, a discectomy procedure may be used to decompress the herniation by accessing the affected disc and removing a portion of the disc and any loose disc fragments. In some cases, a portion of the lamina or bony arch of the vertebrae may be removed. When discectomy fails to resolve a patient's symptoms, more drastic measures may include disc replacement surgery or vertebral fusion.

### BRIEF SUMMARY

In some variations, a tissue removal device may comprise a handheld housing, a motor, and a tissue removal mechanism coupled to the handheld housing. The tissue removal mechanism may comprise a tissue collection chamber coupled to a distal portion of the handheld housing, a jaw member comprising first and second jaw portions, where the first jaw portion is coupled to a first elongated member and the second jaw portion is coupled to a second elongated member configured to actuate the second jaw portion, a rotatable shaft disposed within a lumen of the first elongated member, a helical member disposed around at least a portion of the rotatable shaft, and an impeller coupled to at least one of a distal end of the helical member and a distal end of the rotatable shaft. Rotation of the rotatable shaft may effect rotation of the helical member and the impeller.

In certain variations, a tissue removal device may comprise a handheld housing, a motor, and a tissue removal mechanism coupled to the handheld housing. The tissue removal mechanism may comprise a tissue collection chamber coupled to a distal portion of the handheld housing, a tubular member, a rotatable elongated member disposed within the tubular member; and a tissue removal assembly. The tissue removal assembly may comprise a helical member disposed around at least a portion of the rotatable elongated member, an impeller coupled to a distal portion of at least one of the rotatable elongated member and the helical member, and a distal opening configured to receive and funnel tissue into a narrowed region and toward the impeller. The opening may be bounded by a hood and an inner sheath within the hood, and rotation of the rotatable shaft may effect rotation of the helical member and the impeller.

In certain variations, a method for treating a spinal disc may comprise advancing the above-described tissue removal system to target disc tissue and removing at least a portion of the target disc tissue with the tissue removal system.

Methods of accessing a target site in a patient are also described here. One variation of a method for accessing a target site in a patient may comprise inserting a stylet (e.g. a straight stylet) into a cannula (e.g. a cannula comprising a non-linear configuration), inserting the stylet-cannula assembly into a patient (e.g. where the cannula is at least partially straightened), and removing the stylet from the cannula while substantially maintaining the cannula in the patient. The method may additionally comprise inserting an instrument, such as a tissue removal system, into the cannula.

Methods of accessing a target site in the spine region of a patient are also described here. One variation of a method for accessing a target site in the spine region of a patient may comprise inserting a stylet (e.g. a straight stylet) into a cannula (e.g. a curved cannula with a curved distal portion) to form a first cannula-stylet assembly (e.g. with a straight distal portion). The first cannula-stylet assembly may access the spine region, and the stylet may be proximally withdrawn from the first cannula-stylet assembly. A stylet (e.g. a curved stylet with a curved distal portion) may be inserted into the cannula to form a second cannula-stylet assembly (e.g. with a curved distal portion). The second cannula-stylet assembly may be advanced to the target site in the spine region.

Methods for treating a herniated disc are also described here. One variation of a method for treating a herniated disc may comprise inserting a stylet (e.g. a straight stylet) into a cannula (e.g. a curved cannula with a curved distal portion) to form a first cannula-stylet assembly (e.g. with a straight distal portion). The first cannula-stylet assembly may penetrate the disc annulus of the herniated disc. The stylet may be proximally withdrawn from the first cannula-stylet assembly, and a stylet (e.g. a curved stylet with a curved distal portion) may be inserted into the cannula to form a second cannula-stylet assembly (e.g. with a curved distal portion). The second cannula-stylet assembly may be advanced to a herniated area. The stylet may be proximally withdrawn from the second cannula-stylet assembly, and a tissue removal device may be inserted into the cannula. A portion of the nucleus pulposus may be removed using the tissue removal device. The tissue removal device may be proximally withdrawn from the cannula, and a stylet (e.g. a straight stylet) may be inserted into the cannula. The stylet and the cannula may be proximally withdrawn.

### BRIEF DESCRIPTION OF THE DRAWINGS

FIG. 1 is a schematic perspective view of a portion of a lumbar spine.

FIG. 2 is a schematic superior view of a portion of a lumbar vertebra and disc.

FIG. 3A is a schematic lateral view of a portion of a lumbar spine (without the spinal nerves), and FIG. 3B depicts the portion of the lumbar spine in FIG. 3A (with the spinal nerves depicted).

FIG. 4A is a shaded side view of a variation of a discectomy device; FIG. 4B provides a line drawing of the side view of FIG. 4A; FIG. 4C is a shaded side view of the discectomy device as depicted in FIG. 4A, with a portion of its housing removed; and FIG. 4D provides a line drawing of the side view of FIG. 4C.

FIG. 5A is a shaded exploded view of a portion of the discectomy device of FIG. 4A, and FIG. 5B provides a line drawing of the exploded view of FIG. 5A.

FIG. 6A is an enlarged view of region 6A of the discectomy device of FIG. 4B; FIG. 6B is a top view of the discectomy device region depicted in FIG. 6A; and FIG. 6C is a cross-sectional view of the discectomy device region depicted in FIG. 6A, taken along line 6C-6C.

FIGS. 6D-6F provide an illustrative depiction of the operation of the discectomy device region depicted in FIG. 6A.

FIGS. 6G-6I are illustrative depictions of a variation of a tissue removal assembly of a discectomy device.

FIGS. 6J-6L are illustrative depictions of another variation of a tissue removal assembly of a discectomy device.

FIG. 7A is a shaded illustrative view of a sheath component of the discectomy device of FIG. 4A; FIG. 7B is a front elevational view of the sheath component of FIG. 7A (provided as a line drawing); and FIG. 7C is a side elevational view of the sheath component of FIG. 7A (also provided as a line drawing).

FIG. 8A is a shaded illustrative view of another variation of a sheath component of a discectomy device; FIG. 8B is a front elevational view of the sheath component of FIG. 8A (provided as a line drawing); and FIG. 8C is a side elevational view of the sheath component of FIG. 8A (also provided as a line drawing).

FIG. 9A is a shaded illustrative depiction of a variation of an impeller component of a discectomy device; FIG. 9B is a side elevational view of the impeller component of FIG. 9A (provided as a line drawing); and FIG. 9C is a rear elevational view of the impeller component of FIG. 9A (also provided as a line drawing).

FIG. 10A is a shaded illustrative depiction of another variation of an impeller component of a discectomy device; FIG. 10B provides a line drawing of the shaded illustrative depiction of FIG. 10A; FIG. 10C is a side elevational view of the impeller component of FIG. 10A (provided as a line drawing); FIG. 10D is a top elevational view of the impeller component of FIG. 10A (provided as a line drawing); FIG. 10E is a side elevational view of the impeller component of FIG. 10A (provided as a line drawing); FIG. 10F is an enlarged front elevational view of the impeller component of FIG. 10A (provided as a line drawing); FIG. 10G is an enlarged rear elevational view of the impeller component of FIG. 10A (provided as a line drawing); FIG. 10H is a transverse cross-sectional view of the impeller component of FIG. 10A (provided as a line drawing); and FIG. 10I is a longitudinal cross-sectional view of the impeller component of FIG. 10A (provided as a line drawing).

FIG. 11A is a shaded side view of a variation of a discectomy device comprising a jaw member; FIG. 11B provides a line drawing of the side view of FIG. 11A; FIG. 11C is a shaded side view of the discectomy device as depicted in FIG. 11A, with a portion of its housing removed; and FIG. 11D provides a line drawing of the side view of FIG. 11C.

FIG. 12A is an enlarged view of region 12A of the discectomy device of FIG. 11D; FIG. 12B is an enlarged view of region 12B of FIG. 12A; and FIG. 12C is a rotated enlarged view of region 12B of FIG. 12A.

FIG. 13A is a shaded side view of another variation of a discectomy device comprising a jaw member; FIG. 13B provides a line drawing of the side view of FIG. 13A; FIG. 13C is a shaded side view of the discectomy device as depicted in FIG. 13A, with a portion of its housing removed; FIG. 13D is a line drawing view of the discectomy device as depicted in FIG. 13B, with a portion of its housing removed; FIG. 13E is an enlarged view of region 13E of the discectomy device of FIG. 13B; and FIG. 13F is a shaded side view in partial cross-section of a distal portion of the discectomy device of FIG. 13A.

### DETAILED DESCRIPTION

Tissue removal devices and methods, such as discectomy devices and methods, are described herein. In certain variations, a discectomy device may be introduced into a disc via dilation of an access hole through the annulus, such that it may not be necessary to cut the annulus to access the disc. In some variations, a discectomy device may comprise a relatively long auger, and/or an impeller that breaks down acquired tissue during a procedure. During use, the auger and impeller may effect a plunging motion that allows for relatively rapid tissue aspiration and aggressive tissue cutting, without stretching the annulus. Additionally, it may not be necessary to make several passes into and out of a patient to remove tissue, using devices and methods described herein. By limiting cutting, stretching and/or the number of passes through tissue, scarring of annular tissue, reherniation and/or leakage of healthy nucleus tissue may be avoided, and/or annulus healing time may be reduced.

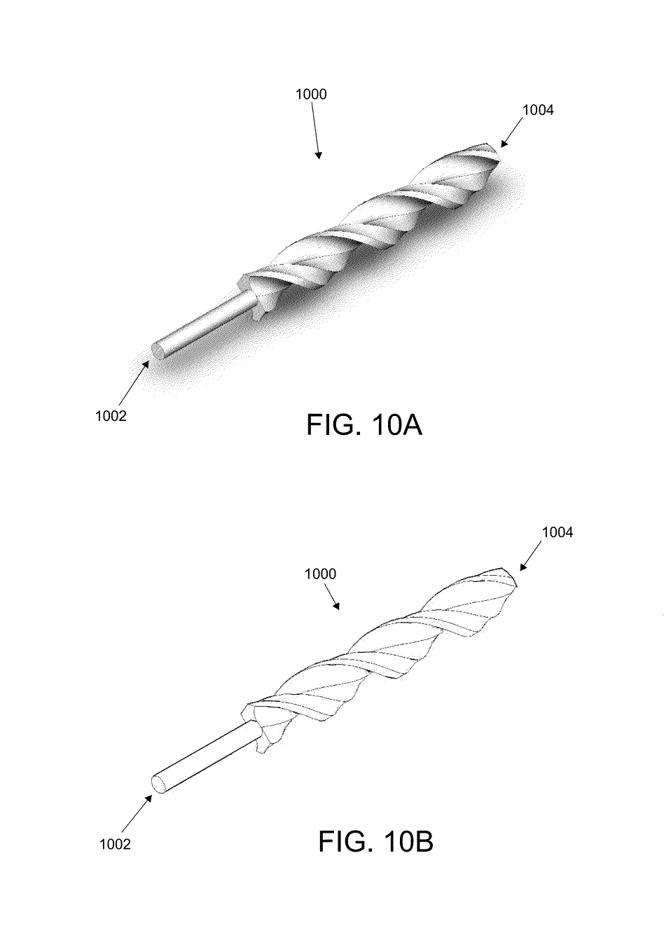

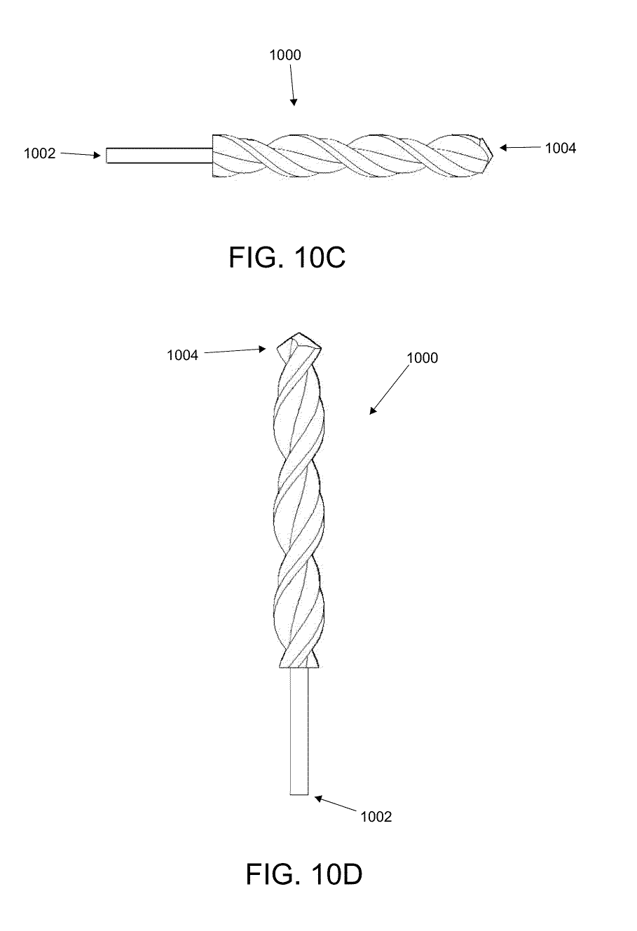

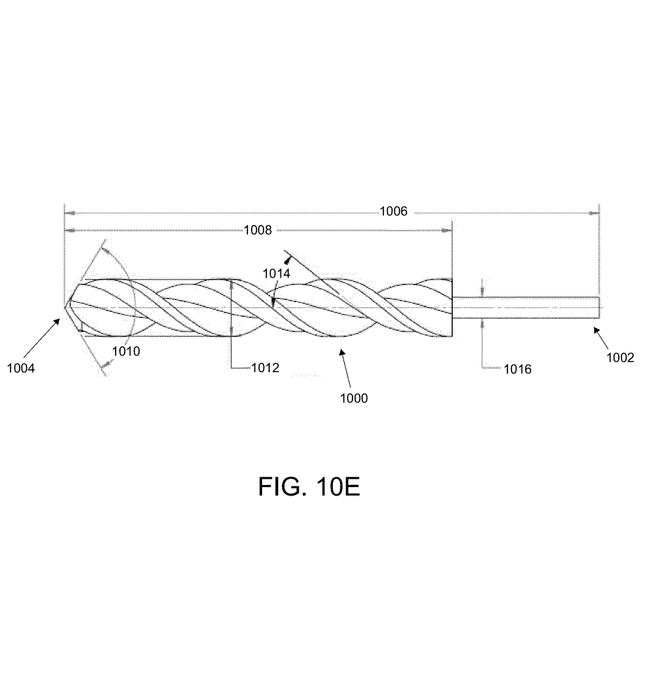

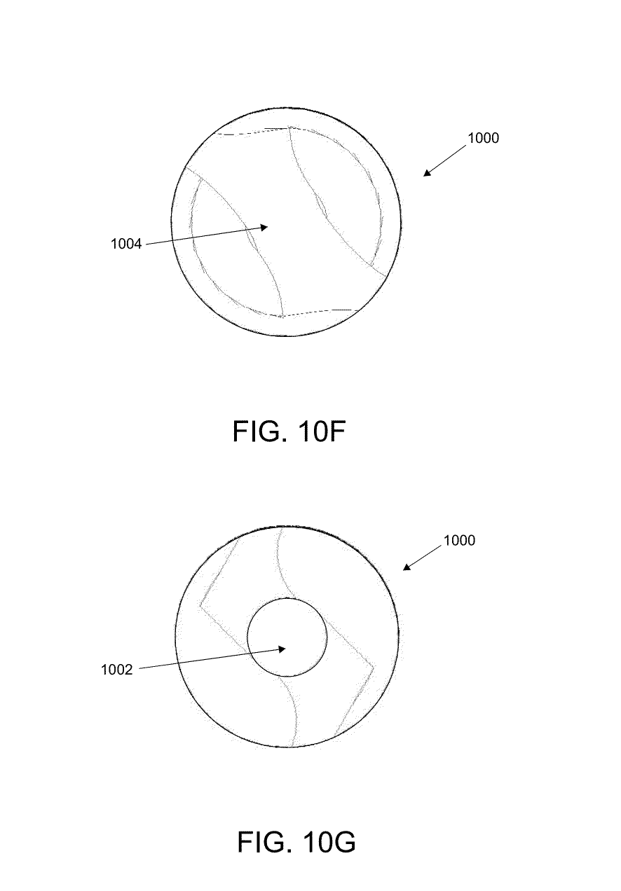

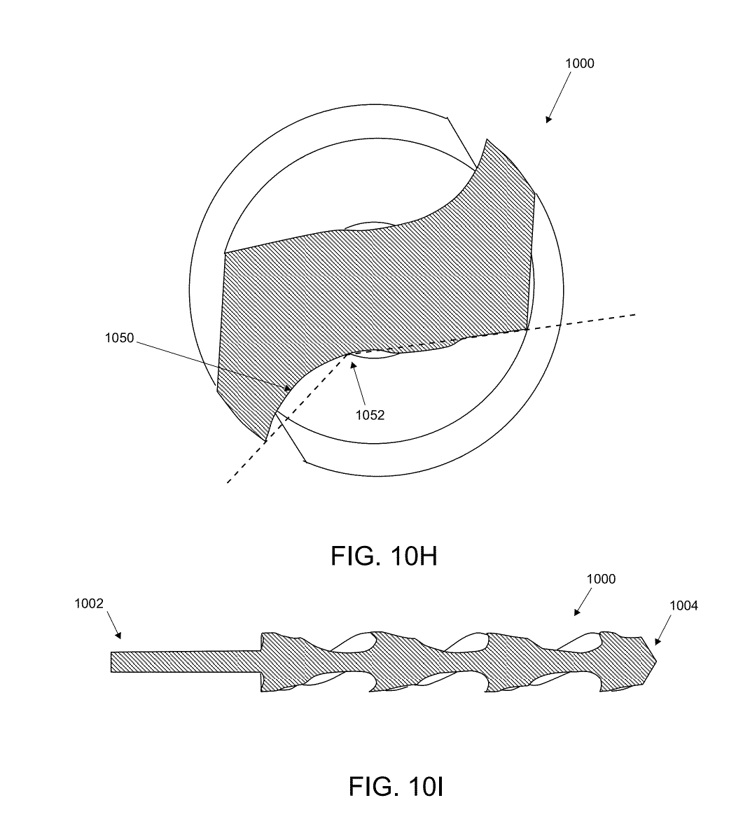

In some cases, devices described herein may be capable of breaking down soft tissue and/or relatively tough, hardened nucleus tissue, and/or may be used to aspirate different types of tissue varying in consistency, hardness and/or elasticity. In some variations, devices described herein may be used to cut hard tissue, such as bone. In some cases, the cant angle of a device's cutting edge or edges (e.g. between the inner base surface of the impeller and the cutting edge of the impeller) may be adjusted to differentially cut relatively hard or calcified materials or tissues without also cutting relatively soft materials or tissues. Examples of these differential cutting heads are described in U.S. Pat. No. 4,445,509, which is hereby incorporated by reference in its entirety. In some cases, a cant angle in the range of about 90 degrees to about 130 degrees may be used. Further discussion of cant angles and cutting edges is provided below with reference to FIGS. 10H and 10I.

In some variations, to be the least destructive to spine structures while preserving the strength of the bones, a spinal procedure may be minimally invasive while also reducing the amount of excised, native bone or dissection of surrounding native tissues. Minimally invasive tissue removal devices may, for example, be configured for insertion toward or into a vertebral disc without requiring suturing, gluing or other procedures to seal or close the access pathway into the disc. The exemplary variations described herein include but are not limited to minimally invasive devices or systems and methods for performing discectomies and other tissue removal procedures, as appropriate. For example, a microdiscectomy may be performed using one or more of the devices and/or methods described herein.

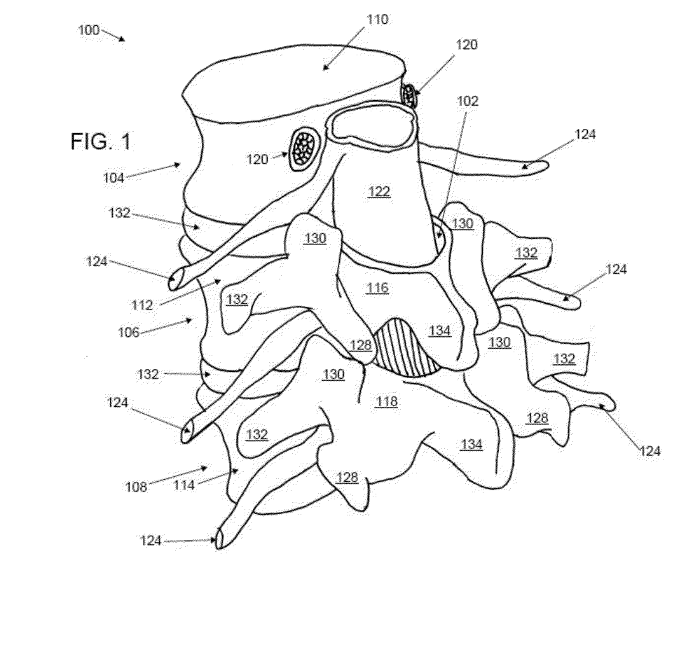

FIG. 1 is a schematic perspective view of a lumbar portion of a spine 100. The vertebral canal 102 is formed by a plurality of vertebrae 104, 106, and 108, which comprise vertebral bodies 110, 112, and 114 anteriorly and vertebral arches 116 and 118 posteriorly. The vertebral arch and adjacent connective tissue of the superior vertebra 104 in FIG. 1 has been omitted to better illustrate the spinal cord 122 within the vertebral canal 102. Spinal nerves 124 branch from the spinal cord 122 bilaterally and exit the vertebral canal 102 through intervertebral foramina 126 that are formed between adjacent vertebra 104, 106 and 108. The intervertebral foramina 126 are typically bordered by the inferior surface of the pedicles 120, a portion of the vertebral bodies 104, 106 and 108, the inferior articular processes 128, and the superior articular processes 130 of the adjacent vertebrae. Also projecting from the vertebral arches 116 and 118 are the transverse processes 132 and the posterior spinous processes 134 of the vertebrae 106 and 108. Located between the vertebral bodies 110, 112 and 114 are vertebral discs 132.

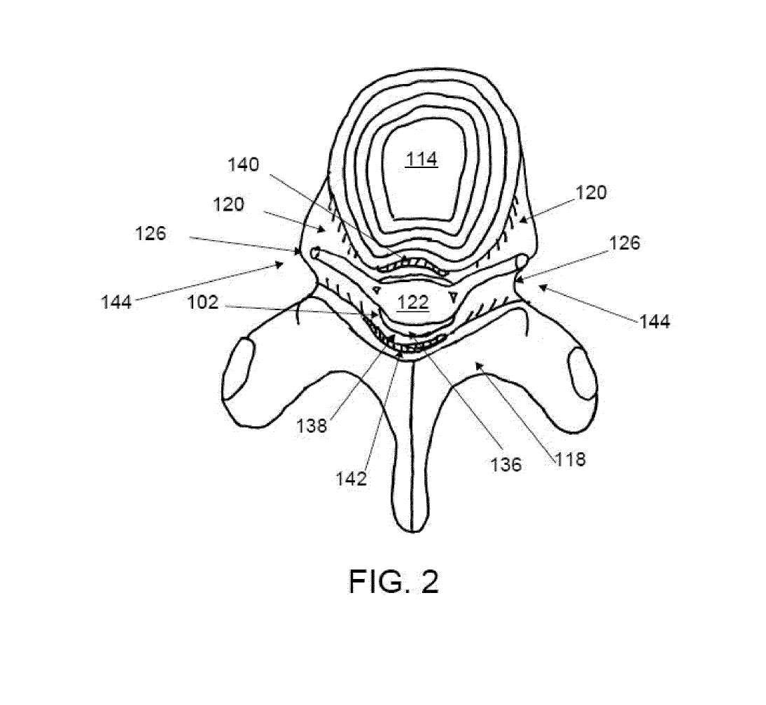

Referring to FIG. 2, the spinal cord 122 is covered by a thecal sac 136. The space between the thecal sac 136 and the borders of the vertebral canal 102 is known as the epidural space 138. The epidural space 138 is bound anteriorly and posteriorly by the longitudinal ligament 140 and the ligamentum flavum 142, respectively, of the vertebral canal 102, and laterally by the pedicles 120 of the vertebral arches 116 and 118 and the intervertebral foramina 126. The epidural space 138 is contiguous with the paravertebral space 144 via the intervertebral foramina 126.

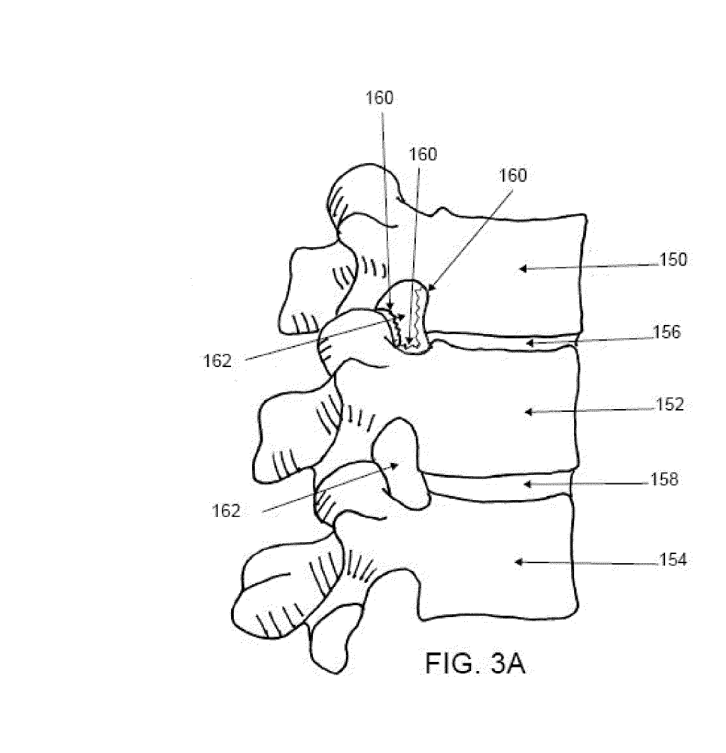

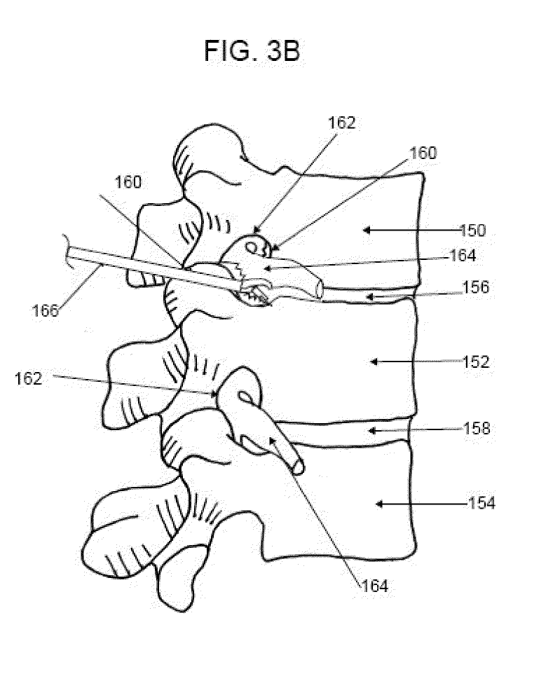

With degenerative changes of the spine, which include but are not limited to disc bulging and hypertrophy of the spinal ligaments and vertebrae, the vertebral canal 102 may narrow and cause impingement of the spinal cord or the cauda equina, a bundle nerves originating at the distal portion of the spinal cord. Disc bulging or bone spurs may also affect the spinal nerves 124 as they exit the intervertebral foramina 126. FIG. 3A, for example, schematically depicts a lateral view of three vertebrae 150, 152 and 154 with intervertebral discs 156 and 158, without the spinal cord or spinal nerves. With degenerative changes, regions of bone hypertrophy 160 may develop about the intervertebral foramina 162. While secondary inflammation of the associated nerve and/or soft tissue may benefit from conservative therapy, the underlying bone hypertrophy remains untreated. The regions of bone hypertrophy 160 may be removed, with or without other tissue, using open surgical spine procedures, limited access spine procedure, percutaneous or minimally invasive spine procedures, or combinations thereof. FIG. 3B depicts the vertebrae 150, 152 and 154 of FIG. 3A with their corresponding spinal nerves 164 during a foraminotomy procedure using a burr or grinder system 166. One example of a limited access spine procedure is disclosed in U.S. Pat. No. 7,108,705, which is hereby incorporated by reference in its entirety. Examples of percutaneous or minimally invasive spine procedures may be found in U.S. Pat. No. 4,573,448, U.S. Pat. No. 6,217,5009, and U.S. Pat. No. 7,273,468, which are hereby incorporated by reference in their entirety.

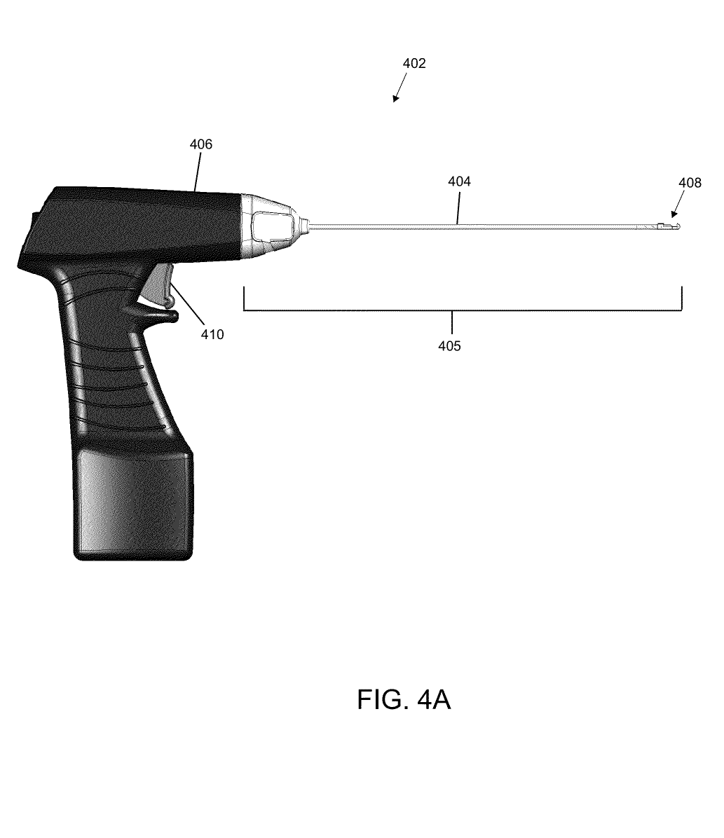

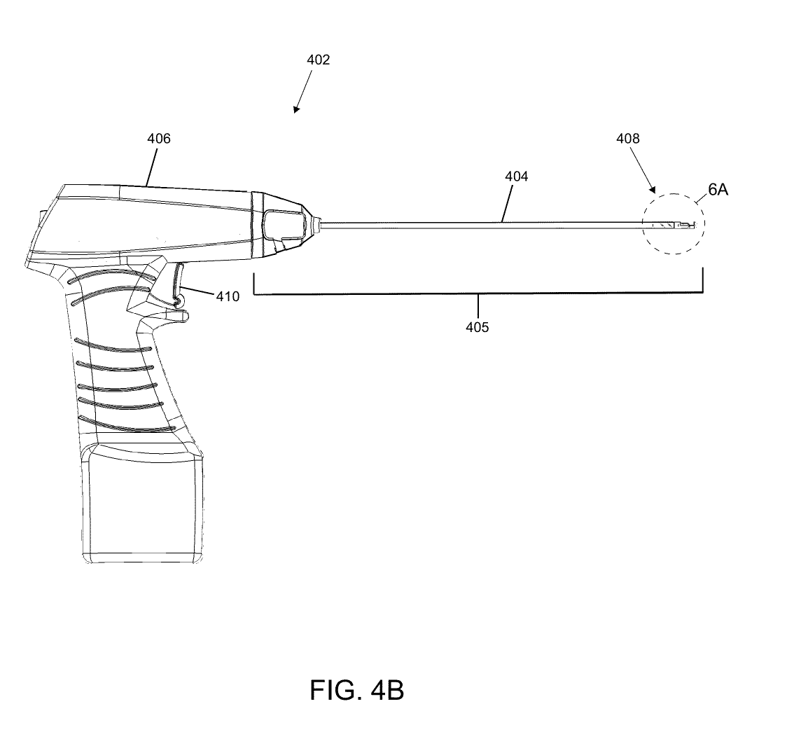

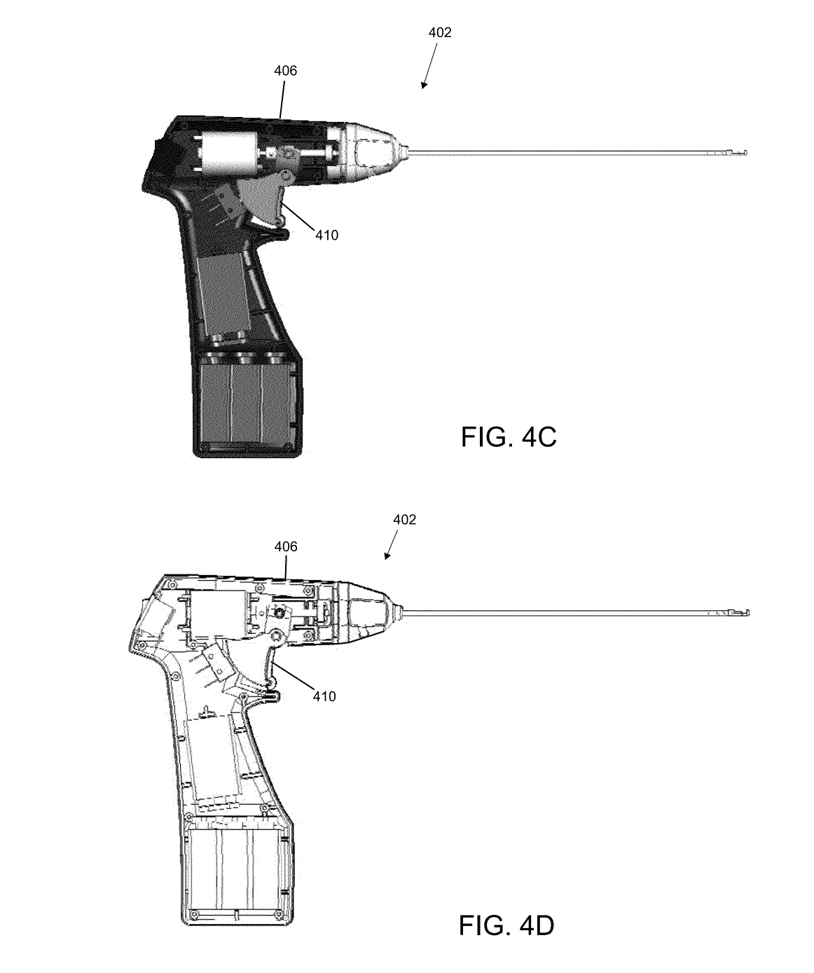

FIGS. 4A-4D depict one variation of a tissue removal device 402, comprising a housing 406 and a tissue removal mechanism 405 comprising (among other components) an outer tube 404 coupled to housing 406. The housing 406 contains one or more components configured to control a tissue removal assembly 408 and other optional features of the tissue removal device 402. The static outer tube 404 covers a rotating drive shaft (not shown) that is attached to the tissue removal assembly 408 of the tissue removal mechanism 405. In other variations, the tissue removal device 402 (and other tissue removal devices described herein, as appropriate) may lack an outer tube and the drive shaft of the tissue removal device may be inserted into a lumen of a cannula or other access device. Outer tube 404 may comprise any suitable material or materials, such as metals and/or metal alloys, such as stainless steel. In some variations, the material or materials used for the outer tube may be selected to achieve a desirable balance between stiffness and flexibility. It should be understood from the outset that features and/or characteristics of tissue removal devices and methods described herein may be applied to other tissue removal devices and methods (including others described herein), as appropriate.

Tissue removal assemblies such as tissue removal assembly 408, may be configured to grasp, cut, chop, grind, burr, pulverize, debride, debulk, emulsify, disrupt or otherwise remove tissue, as appropriate. Emulsification includes, for example, forming a suspension of tissue particles in a medium, which may be the existing liquid at the target site, liquid added through the tissue removal device, and/or liquid generated by the debulking of the tissue. Optional components of tissue removal device 402 and other tissue removal devices described herein may include, but are not limited to, a motor configured to rotate or move one or more components of the tissue removal assembly, a power source or power interface, a motor controller, a tissue transport assembly (e.g. comprising an auger), an energy delivery or cryotherapy assembly, a therapeutic agent delivery assembly, a light source, and one or more fluid seals. The optional tissue transport assembly may comprise a suction assembly and/or a mechanical aspiration assembly. One or more of these components may act through the outer tube 404 to manipulate the tissue removal assembly and/or other components located distal to the housing 406, or from the housing 406 directly. For example, the tissue removal device 402 may further comprise an optional port that may be attached to an aspiration or suction source to facilitate transport of tissue or fluid out of the target site or patient. The suction source may be a powered vacuum pump, a wall suction outlet, or a syringe, for example.

The housing 406 may further comprise a control interface 410 that may be used to control the power state of the tissue removal device 402, including but not limited to on and off states. In this particular variation, the control interface 410 comprises a trigger that may be squeezed to operate the device, but in other variations, control interface 410 may comprise a push button, a slide, a dial, a knob, a lever or a pivot member, for example. In some variations, control interface 410 may also change the motor speed and/or movement direction of the tissue removal assembly 408. A bi-directional tissue removal device may be provided, for example, as a potential safety feature should the tissue removal assembly 408 get lodged in a body tissue or structure. The web-like connective tissue that may be found in the epidural space may get wound onto or caught up on the tissue removal assembly. This connective tissue may be dislodged with a bi-directional tissue removal device by reversing the direction of rotation to unwind the tissue. The control interface 410 may be analog or digital, and may comprise one or more detent positions to facilitate selection of one or more pre-selected settings. In other variations, a separate motor control interface may be provided for one or more features of the motor. In still other variations, control interfaces for other features of the tissue removal device may be provided.

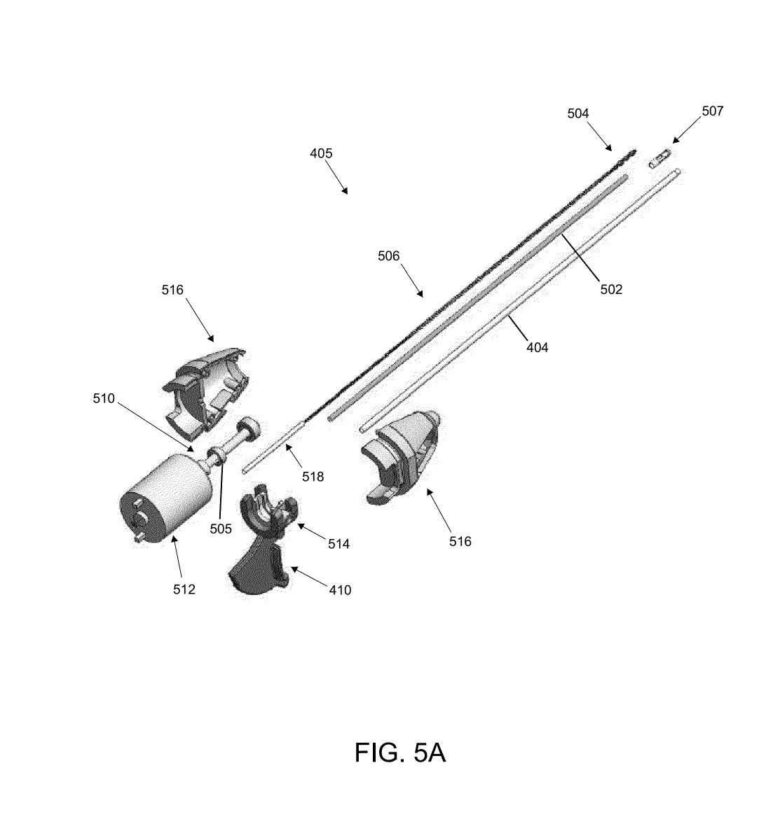

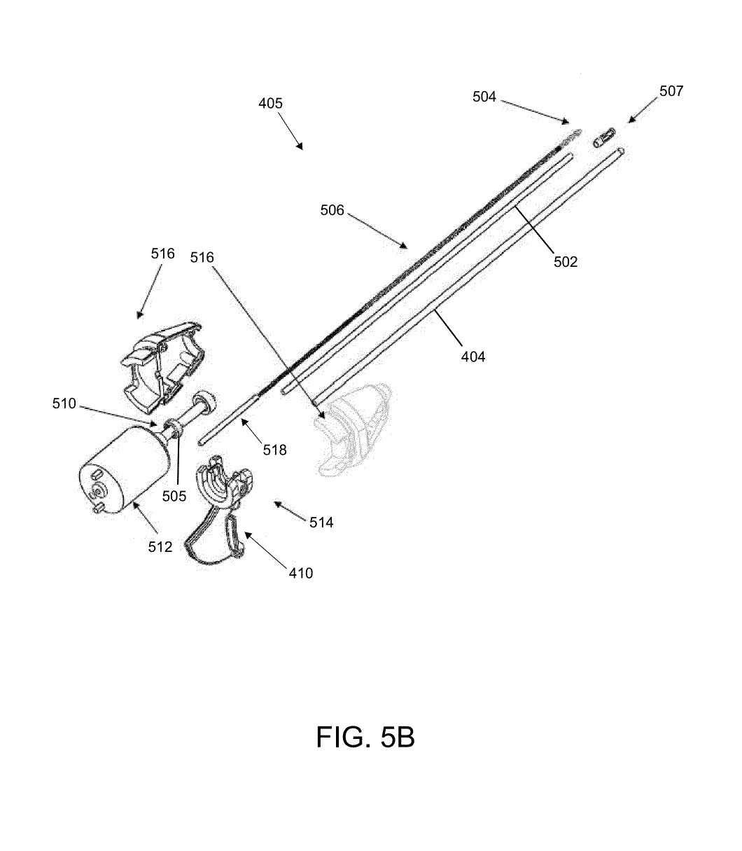

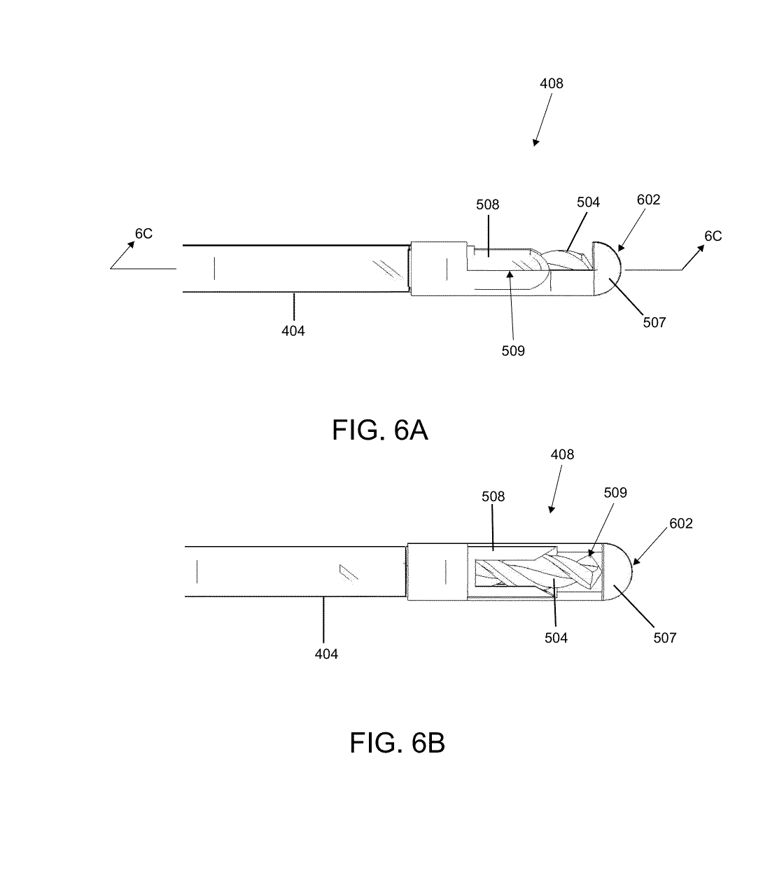

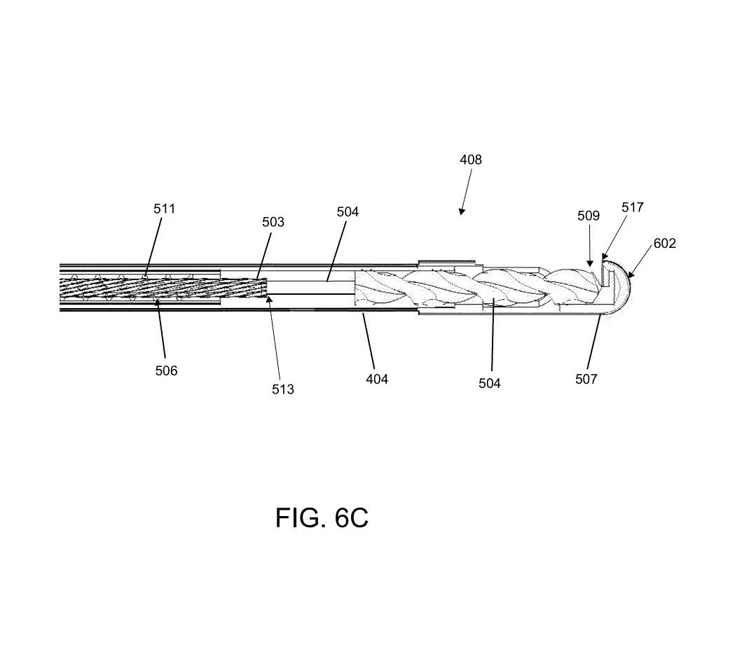

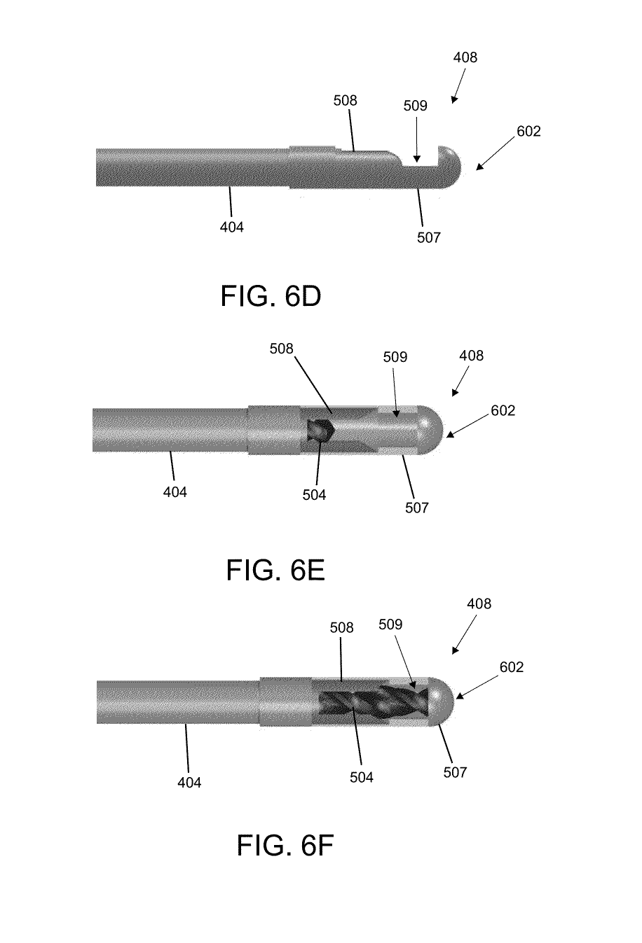

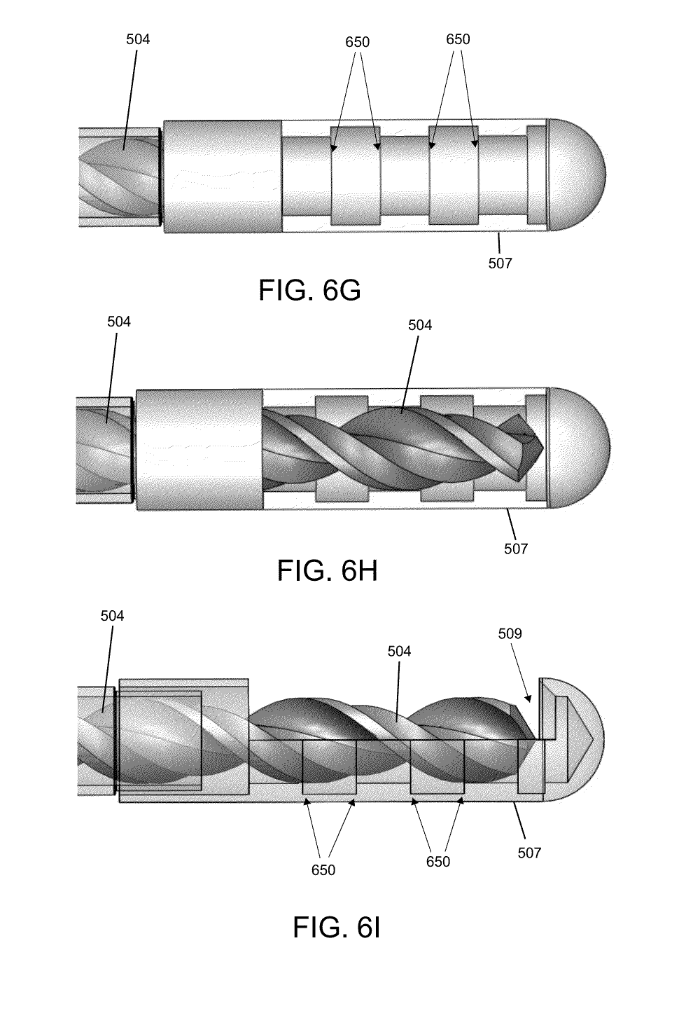

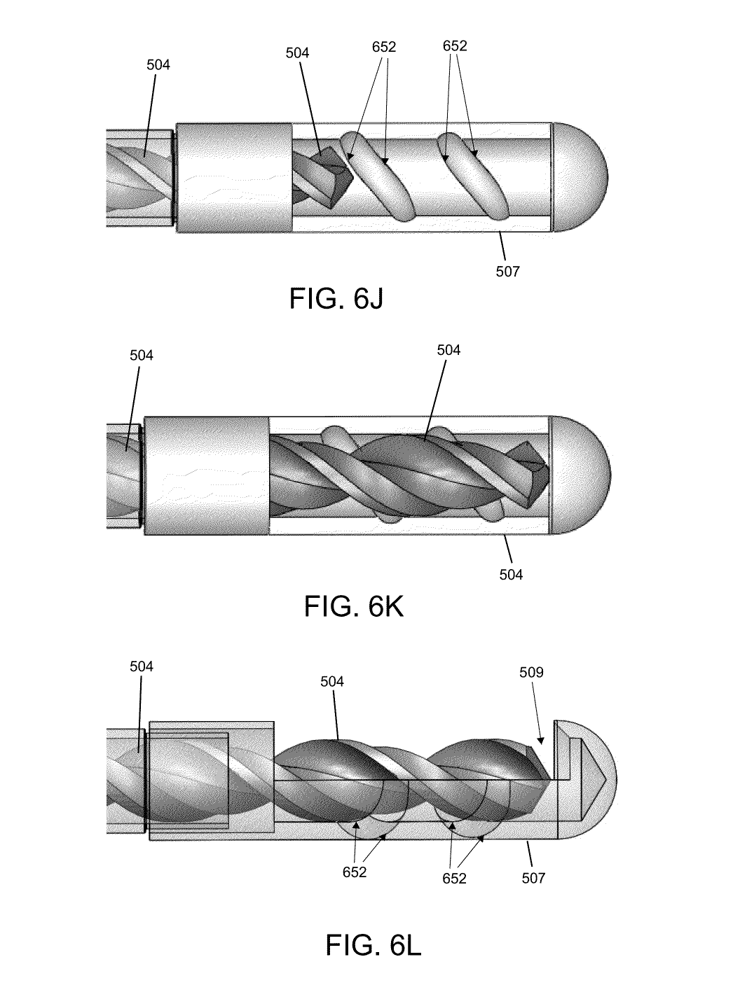

Referring to FIGS. 5A, 5B and 6A-6F, the tissue removal assembly 408 may comprise a tissue transport assembly 506 comprising a drive shaft 503 (FIG. 6C) and a helical member or auger 511 (FIG. 6C) coupled to the drive shaft 503. Additionally, the tissue removal assembly 408 may comprise an impeller 504 that, as shown here, may be coupled (e.g. welded) to a distal end 513 (FIG. 6C) of drive shaft 503. Tissue removal assembly 408 may further comprise a hooded tip 507 housing a sheath 508, where the sheath 508 comprises an aperture 509. Impeller 504 is disposed within sheath 508, but is partially exposed as a result of the presence of aperture 509.

In some cases, hooded tip 507 may comprise a backward- or proximally-facing edge (e.g. edge 517 in FIG. 6C) that may provide for additional tissue scraping and/or removing capabilities without, for example, increasing the likelihood of unintentionally perforating a disc annulus or gouging an end-plate. The tissue removal edges of hooded tip 507 may act in conjunction with impeller 504, or may be used to manually by the operator to remove tissue, e.g. manipulating the hooded tip 507 in a back and forth motion to remove tissue.

In certain cases, a hooded tip 507 (e.g. the edge of an opening or aperture of the hooded tip) may include one or more other features, such as grooves, channels, sharpened or serrated configurations, or the like, that may be used to further enhance tissue cutting and maceration (e.g. by acting as a static cutting edge). In some cases, the interior surface of a hooded tip 507 may alternatively or additionally comprise one or more protrusions, recesses and/or other cutting structures to facilitate further tissue disruptions.

For example, FIGS. 6G-6I depict a hooded tip 507 comprising shearing edges 650 formed by grooves in the inside surface of the hooded tip. Shearing may be implemented in a hooded tip or in jaws (which are described further below) by, for example, adding such grooves and/or similar features to the interior surface of the hooded tip or jaws. The grooves may replace the sheath 508 (as a separate component) to provide a shearing effect between the shearing edges and the impeller 504, or may be included in addition to the sheath 508. Examples of suitable shearing edges may include straight grooves or angled grooves or a cross-helical pattern (as shown, for example, in FIGS. 6J-6L, depicting angles grooves 652). The angled grooves may, for example, be located at an opposing angle to the flutes to provide a good shearing edge.

Referring specifically now to FIGS. 5A and 5B, tissue removal assembly 408 may comprise a liner 502 (e.g. comprising polyimide) that may, for example, help to prevent heat transfer and/or grinding between metal tissue removal assembly components. Additionally, tissue removal mechanism 405 may further comprise a driveshaft coupler 510, as well as a motor 512 configured to cause the rotatable drive shaft 503 to rotate. Additionally, the tissue removal mechanism 405 may comprise a trigger housing saddle 514 that operably couples the trigger 410 to the components of the tissue removal assembly 408, and a collection chamber 516 for collecting tissue during use.

When tissue removal device 402 is operated, auger 511 may advance into and retract from sheath 508, with impeller 504 acting as a forward “drilling” cutter and also as a side cutter, as a result of the impeller's interactions with the sheath 508 housed in the hooded tip 507. Auger 511 may be actuated by pulling on trigger 410, where saddle 514 slides a proximal bearing 505 along a slot (not shown) of the driveshaft coupler 510. The bearing 505 is coupled to a proximal portion 518 of the tissue transport assembly 506 (e.g. auger 511), such that the auger 511 may slide freely back and forth in the driveshaft coupler 510. The hooded tip 507, which may be similar to a half jaw, half guards the impeller 504 and the sheath 508. This may control which side is performing the cutting, and may also provide a relatively tight clearance shearing surface along the length of impeller 504. In some variations, there may be a relatively tight gap (e.g. 0.0005 inch to 0.002 inch) between impeller 504 and the sheath 508 or the internal surface of rounded distal head 602 (described in additional detail below). This relatively tight gap may, for example, provide for good shearing.

Auger 511 may be used to facilitate transport or removal of tissue within or along outer tube 404. In the particular variation depicted, auger 511 is mounted on rotatable drive shaft 503, and is also capable of moving axially. Actuation of the rotatable shaft 503 may mechanically facilitate proximal movement of tissue or other materials within the channel or the lumen of the outer tube 404 by rotating auger 511. The actuated rotatable shaft 503 will also rotate impeller 504. In some variations, use of tissue transport assembly 506 may be performed at lower rotational speeds when tissue debulking is not concomitantly performed. When rotated in the opposite direction, the auger 511 may be used expel or distally transport tissue, fluid or other materials or agents from the outer tube 404 or supplied to an infusion port of the housing 406. While outer shaft 404 and hooded tip 507 may generally be fixed with respect to the rotating auger 511 and impeller 504, in some variations outer shaft 404 and/or hooded tip 507 may be rotatable (e.g. to rotationally orient aperture 509). In some variations, a rotation handle (not shown), such as a knob, may be attached to the proximal end of a movable outer shaft that is distal to the housing to facilitate movement. A locking mechanism may also be provided to resist undesied motion of the shaft after placement in the desired orientation.

In some variations, auger 511 may have a longitudinal dimension of about 6 inches to about 15 inches (e.g. about 6 inches to about 12 inches). In other variations, the longitudinal dimension of the auger 511 may be characterized as a percentage of the longitudinal dimension of the outer tube 404, and may range from about 5% to about 100% of the longitudinal dimension of outer tube 404, sometimes about 10% to about 50%, and other times about 15% to about 25%, and still other times about 5% to about 15%. Although the auger 511 depicted in FIG. 6C rotates at the same rate as the rotatable drive shaft 503 and the impeller 504, in other variations, the auger 511 may be configured to rotate separately from other device components. For example, an auger may comprise a helical coil that is located along at least a proximal portion of the lumen of the outer tube but is not mounted on a rotatable shaft. In this particular example, the auger may rotate independently a shaft (e.g. a rotatable shaft). In still other variations, the auger 511 may be mounted on the surface of the lumen of the outer tube 404, and may be used to transport tissue or substances along the lumen by rotation of the outer tube 404, independent of the rotatable drive shaft 503 or certain other components.

Although auger 511 is depicted as a continuous structure, in some variations, auger 511 may be interrupted at one or more locations. Also, the degree or angle of tightness of the auger 511 may vary, from about 0.5 turns/mm to about 2 turns/mm, sometimes about 0.75 turns/mm to about 1.5 turns/mm, and other times about 1 turn/mm to about 1.3 turns/mm. The cross-sectional shape of the auger 511 may be generally rounded, but in other variations, may have one or more edges. The general cross-sectional shape of the auger 511 may be circular, elliptical, triangular, trapezoidal, squared, rectangular or any other shape. The turn tightness and cross-sectional shape or area of the auger 511 may be uniform or may vary along its length. In some variations, multiple augers may be provided in parallel or serially within the outer tube.

In the various examples described herein, the outer tube and the driveshaft or rotatable shaft of the tissue removal device may comprise a rigid structure and material, but may also optionally comprise at least one flexible region which may bend while still permitting rotation of the driveshaft. Examples of flexible driveshafts that may be used are disclosed in U.S. Pat. Nos. 5,669,926 and 6,053,907, which are hereby incorporated by reference in their entirety. In some examples, the flexible region(s) may comprise a substantial portion or all of the length of the driveshaft and outer tube. A tissue removal device with a flexible region may facilitate access to certain regions of the body, such as the central spinal canal through an intervertebral foramen. In some examples, the flexible tissue removal device may comprise a steering assembly that uses one or more steering wires that are attached distal to the flexible region and manipulated by a steering member in the proximal housing. Other steering mechanisms used with catheters and other elongate instruments may also be used. In other examples, an active steering mechanism is not provided on the flexible tissue removal device, but the flexible tissue removal device may be steered by an endoscopic instrument into which the tissue removal device has been inserted. Some examples of steerable endoscopic instruments are disclosed in U.S. patent application Ser. No. 12/199,706, which is hereby incorporated by reference in its entirety.

FIGS. 6D-6F show tissue removal assembly 408 in use, with FIGS. 6D and 6E depicting side and top views, respectively, of the assembly before impeller 504 has been distally advanced, and FIG. 6F providing a top view of tissue removal assembly 408 after impeller 504 has been distally advanced.

During use, the tissue removal assembly 408 may, for example, be introduced into a disc via dilation through the annulus, such that no annular tissue is cut. The tissue removal assembly 408 and any other device components that pass through the annular tissue may have a maximum outer diameter of, for example, about 2 millimeters to about 4 millimeters (e.g. about 3 millimeters to about 4 millimeters). This may allow the access hole to be dilated to a size where healing and sealing of the annulus can occur more easily than may be the case with a cut annulus.

Tissue removal device 402 may allow for a relatively significant extension of auger 511, with impeller 504 working at grabbing and aspirating tissue. The impeller 504 may work to break down acquired tissue in conjunction with a shearing or cutting edge, such as a tip of the sheath 508. The device 402 may not stretch the disc annulus, as passes of the tissue removal mechanism 405 may not be required. For example, the auger 511 may effectively plunge back and forth so that the impeller 504 may drill through the target tissue. This auger/impeller “plunging” may allow for relatively quick tissue aspiration and/or additional significant cutting into the tissue.

Tissue removal assembly 408, which is shown in further detail in FIGS. 6A-6F, comprises a rounded distal head 602. This rounded distal head may serve as a guard that, for example, prevents inadvertent cutting into a vertebral end-plate or an annulus during use. Additionally, the internal edge of the rounded distal head may comprise a chamfer. The chamfer may, for example, provide a controlled scraping edge that can act as a curette and help pull disc nucleus toward impeller 504 without cutting into a vertebral end-plate or an annulus.

While tissue removal assembly 408 comprises a rounded distal head 602, other head configurations are also contemplated, including but not limited to a conical configuration, an ovoid configuration, a dome configuration, a concave configuration, a cube configuration, etc. The head 602 may be configured to penetrate or dissect body tissue, such as the annular wall of a vertebral disc, and may be used while the rotatable shaft is being rotated, or when the rotatable shaft is not rotated. In other embodiments, the head may comprise multiple points or edges that may be used to cut, chop, grind, burr, pulverize, debride, debulk, emulsify, disrupt or otherwise remove tissue or body structures. In still other embodiments, the head may comprise surfaces with a grit that may be used as a burr mechanism. The grit number may range from about 60 to about 1200 or more, sometimes about 100 to about 600, and other times about 200 to about 500.

The head may optionally comprise a port or aperture which may be used to perform suction or aspiration at the target site and/or to perfuse saline or other biocompatible fluids or materials to the target site. Use of saline or other cooling materials or liquids, for example, may be used to limit any thermal effect that may occur from frictional or other forces applied to the target site during removal procedures. The saline or other materials may or may not be chilled. In other variations, one or more therapeutic agents may be provided in the saline or fluid for any of a variety of therapeutic effects. These effects may include anti-inflammatory effects, anti-infective effects, anti-neoplastic effects, anti-proliferative effects, hemostatic effects, etc.

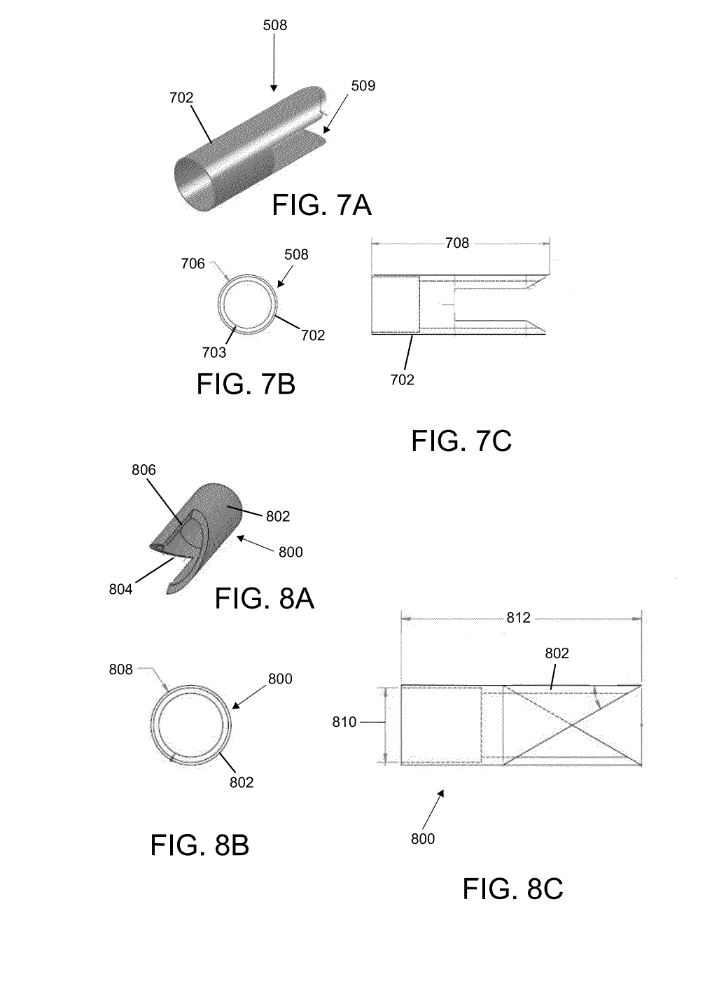

FIGS. 7A-7C depict sheath 508 in enlarged detail. As shown there, sheath 508 comprises a wall portion 702, with aperture 509 formed in the wall portion. Additionally, and referring specifically to FIGS. 7B and 7C, sheath 508 has an inner diameter 704, an outer diameter 706, and a length 708. In some variations, inner diameter 704 may be from about 0.08 inch to about 1.0 inch (e.g. 0.0935 inch), outer diameter 706 may be from about 0.07 inch to about 0.13 inch (e.g. 0.1 inch), and/or length 708 may be from about 0.2 inch to about 0.4 inch (e.g. 0.3 inch). Also, referring to FIG. 7B, inner circle 703 identifies a space within sheath 508 that may be occupied by impeller 504 during use. Sheath 508 may be formed of any appropriate material or materials, including but not limited to polymers, metals and/or metal alloys (e.g. stainless steel, such as 17-4 stainless steel).

Other sheath configurations may also be used. For example, FIGS. 8A-8C depict a sheath 800 comprising a wall portion 802 having two apertures or cut-outs 804 and 806 formed therein, thereby forming an angled sheath tip. Additionally, and referring specifically to FIGS. 8B and 8C, sheath 800 has an inner diameter 810, an outer diameter 808, and a length 812. In some variations, inner diameter 810 may be from about 0.08 inch to about 1.0 inch (e.g. 0.0935 inch), outer diameter 808 may be from about 0.07 inch to about 0.13 inch (e.g. 0.1 inch), and/or length 812 may be from about 0.2 inch to about 0.4 inch (e.g. 0.3 inch). Sheath 800 may be formed of any appropriate material or materials, including but not limited to polymers, metals and/or metal alloys (e.g. stainless steel, such as 17-4 stainless steel). While certain configurations of sheaths have been depicted here, other suitable sheath configurations may also be used in some variations.

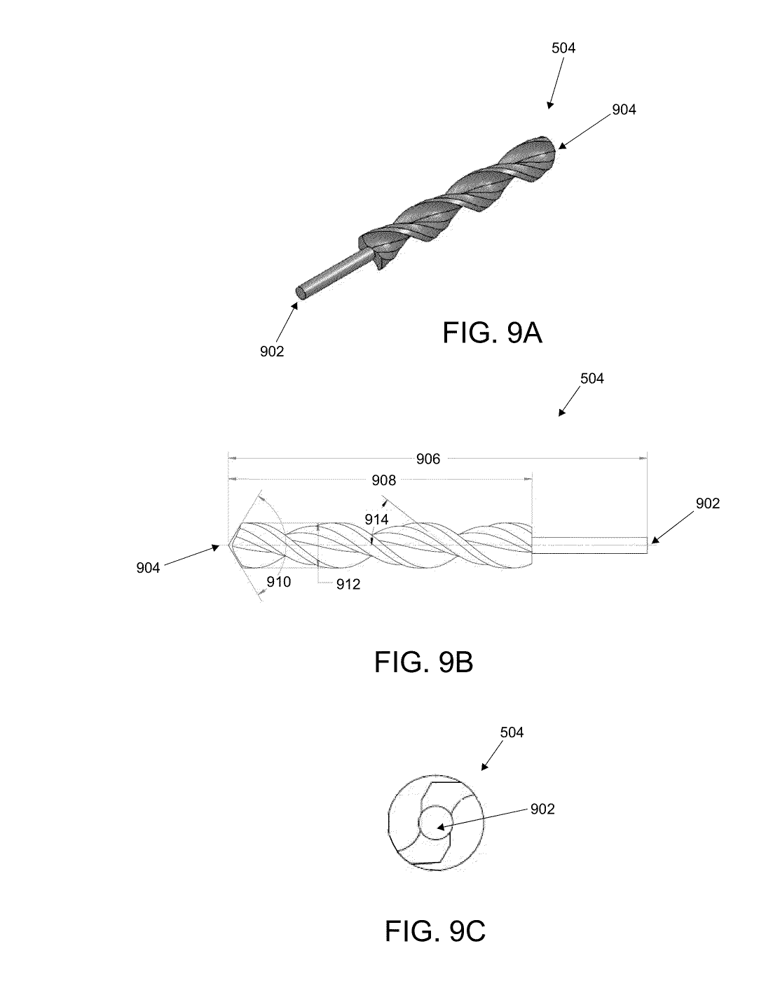

FIGS. 9A-9C depict impeller 504 in additional detail. As shown there, impeller 504 resembles a parabolic drill bit, with a proximal end 902, a distal end 904, and a relatively large relief cut into the back section of its flutes to allow for clearance and better flow of material as the material is drilled out. The impeller 504 also may have forward cutting abilities with a pointed distal end 904 and/or open flutes at the distal end or tip 904, which may aid in centering the impeller.

Referring specifically to FIG. 9B, impeller 504 has dimensions 906, 908, 910, 912 and 914. In some variations, dimension 906 may be from about 0.6 inch to about 0.8 inch (e.g. 0.726 inch), dimension 908 may be from about 0.4 inch to about 0.6 inch (e.g. 0.526 inch), dimension 910 may be from about 105 degrees to about 130 degrees (e.g. 118 degrees), dimension 912 may be from about 0.7 inch to about 0.9 inch (e.g. 0.785 inch), and/or dimension 914 may be from about 36 degrees to about 40 degrees (e.g. 38 degrees). Impeller 504 may, for example, have a pitch (or distance between two adjacent revolutions of the impeller) of about 0.2 inch to about 0.4 inch (e.g. 0.3 inch). In certain variations, the length of the helical path of the impeller may be about 0.7 inch to about 1.5 inches (e.g. 1.12 inches). Impellers may be made of any appropriate material or materials, including but not limited to metals and/or metal alloys such as stainless steel (e.g. 17-4 PH H900 stainless steel).

Other variations of impellers having different configurations may also be used. For example, FIGS. 10A-10I depict an impeller 1000 comprising a proximal end 1002 and a distal end 1004. Referring specifically to FIG. 10E, impeller 1000 has dimensions 1006, 1008, 1010, 1012, 1014 and 1016. In some variations, dimension 1006 may be from about 0.6 inch to about 0.8 inch (e.g. 0.726 inch), dimension 1008 may be from about 0.4 inch to about 0.6 inch (e.g. 0.526 inch), dimension 1010 may be from about 105 degrees to about 130 degrees (e.g. 118 degrees), dimension 1012 may be from about 0.7 inch to about 0.9 inch (e.g. 0.785 inch), dimension 1014 may be from about 37 degrees to about 41 degrees (e.g. 39 degrees), and/or dimension 1016 may be from about 0.02 inch to about 0.04 inch (e.g. 0.028 inch).

It should be understood that features of the above-described impellers and/or other components described herein may be applied to other impellers and/or components of tissue removal devices, as appropriate.

Other variations of tissue removal devices having different configurations may alternatively or additionally be used during a tissue removal procedure, such as a discectomy. In some cases, a pituitary rongeur or other device having jaws and/or jaw-like features may be used in a tissue removal procedure.

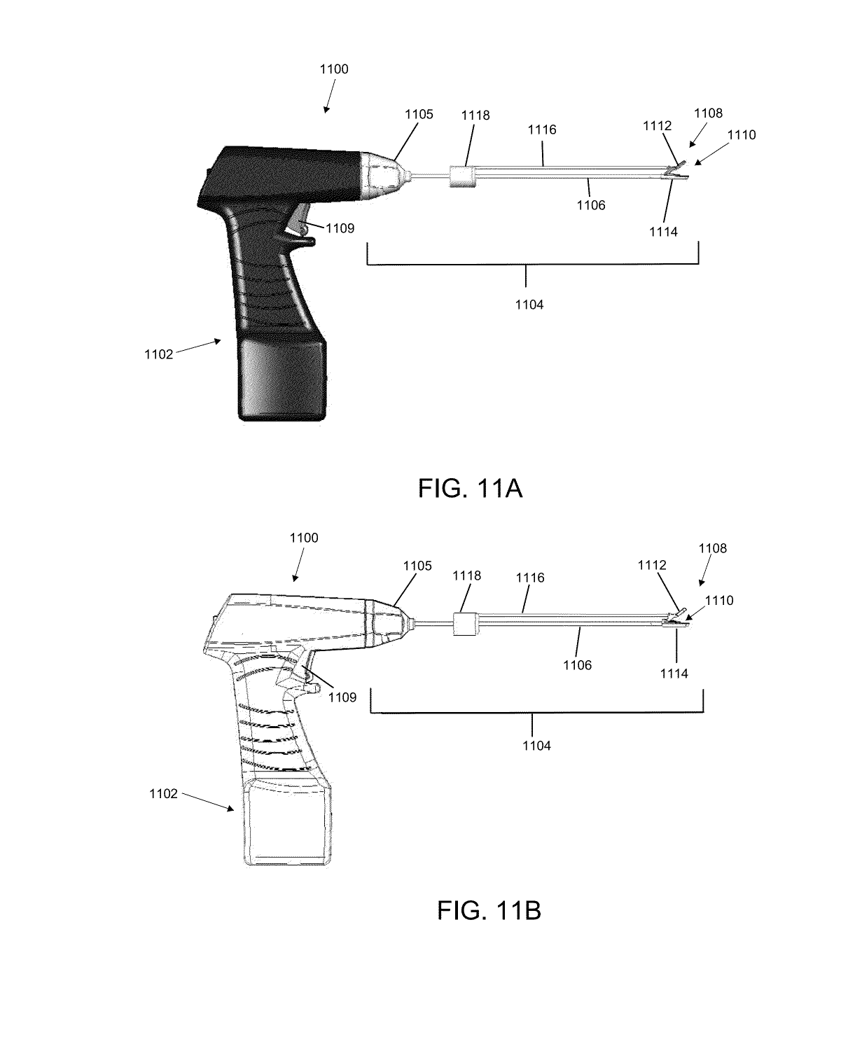

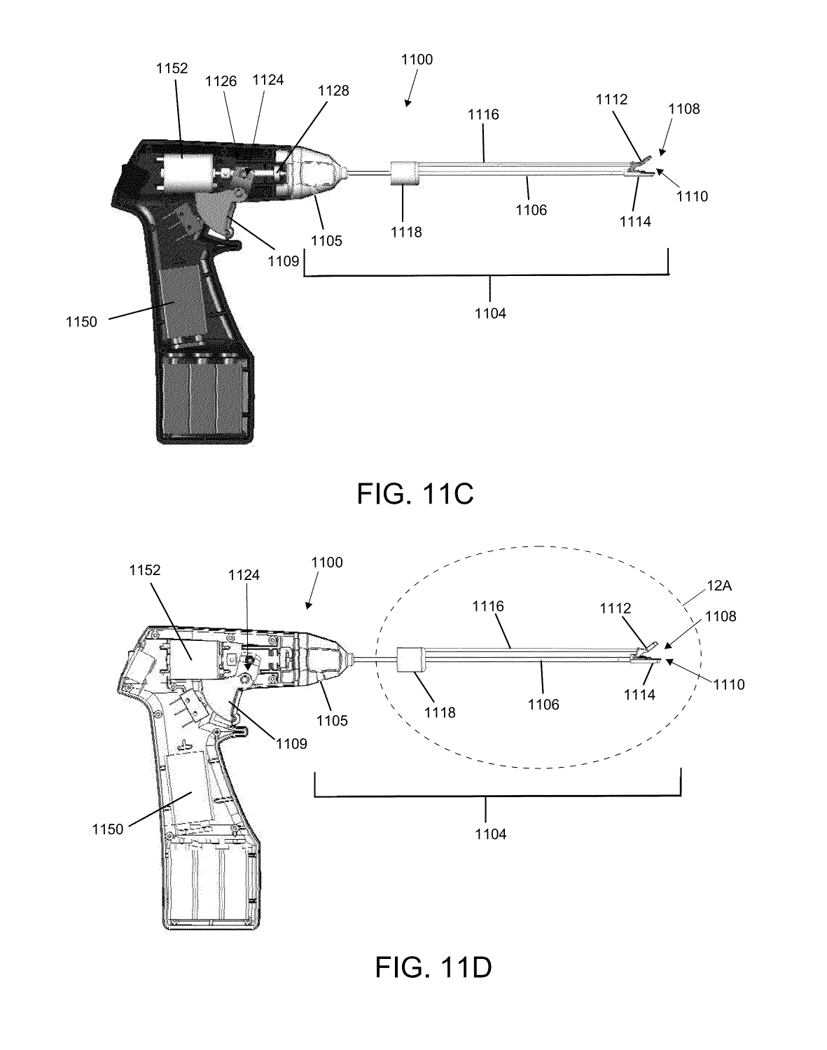

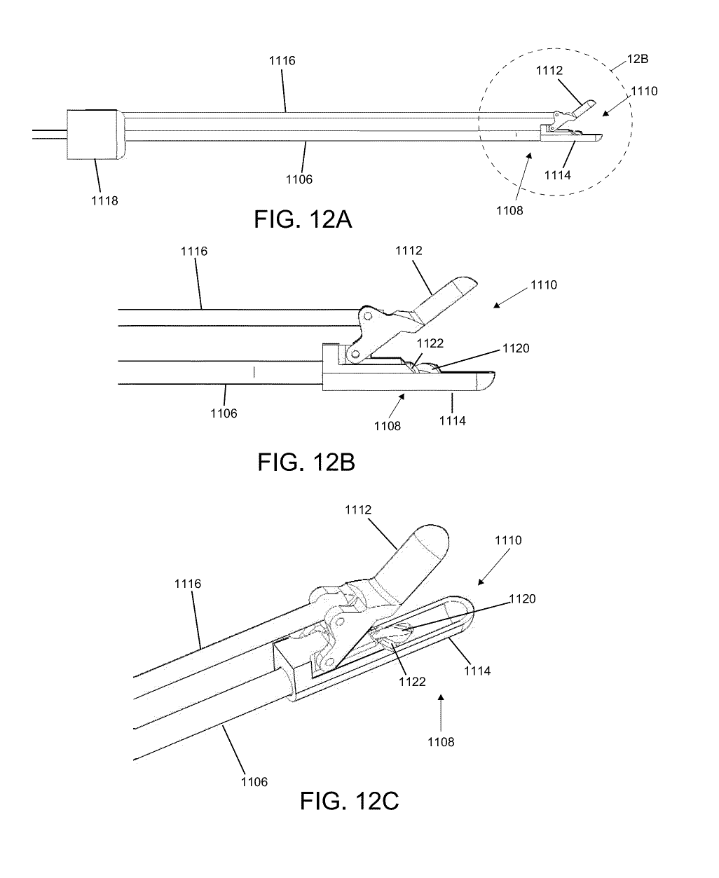

One example of a tissue removal device comprising jaws is depicted in FIGS. 11A-11D and 12A-12C. As shown there, a tissue removal device 1100 comprises a housing 1102 and a tissue removal mechanism 1104 comprising (among other components) a tubular member 1106 coupled to housing 1102, as well as a tissue collection chamber 1105. The tubular member 1106 is coupled to a tissue removal assembly 1108 (depicted in additional detail in FIGS. 12A-12C) of the tissue removal mechanism 1104. The housing 1102 may, for example, contain one or more components configured to control the tissue removal assembly 1108 and other optional features of the tissue removal device 1100. As shown, housing 1102 has a handle configuration that may, for example, be ergonomically designed. A trigger 1109 that may be used to actuate tissue removal device 1100 is coupled to the housing 1102. Trigger 1109 is coupled to a trigger extension 1124 (FIGS. 11C and 11D) contained within housing 1102.

In FIGS. 11C and 11D, tissue removal device 1100 is illustrated with a portion of the housing 1102 removed to show various internal components. In this variation, the tissue removal device 1100 further comprises a battery 1150 to provide power to the motor 1152 which drives the tissue removal assembly 1108. In other variations, a connector to an external power source may be provided in addition to, or in lieu of, the battery 1150. The type of battery and power provided may differ depending upon the particular power needs of the motor and/or other components of the tissue removal device 1100.

In some variations, the motor 1152 of the tissue removal device 1100 is a DC motor, but in other variations, the motor 1152 may have any of a variety of configurations, including but not limited to an AC or a universal motor. The motor 1152 may be a torque, brushed, brushless or coreless type of motor. In some variations, the motor 1152 may be configured to provide a rotational speed of about 500 rpm to about 200,000 rpm or more, sometimes about 1,000 rpm to about 40,000 rpm, and other times about 5,000 rpm to about 20,000 rpm. The motor 1152 may act on the tissue removal assembly 1108 via the tubular member 1106, or a by drive member located within tubular member 1106. In some further embodiments, a fluid seal may be used to protect the motor 1152 and/or other components of the housing 1102 from any fluids or other materials that may be transported through tubular member 1106. In some variations, housing 1102 may be configured to be coupled to a trocar, an introducer, a cannula or another tubular member into which the tissue removal assembly 1108 and the tubular member 1106 are inserted during use. In certain variations, the tissue removal device may be used with an introducer or cannula having an outer diameter of about 0.1 cm to about 1.5 cm or more, sometimes about 0.1 cm to about 1 cm, and other times about 2 mm to about 6 mm.

In some variations, a housing of a tissue removal device, such as housing 1102, may be configured with a size and/or shape that permits handheld use of the tissue removal device. In other variations, the tissue removal device may comprise a grip or structure (e.g. located about a tubular member of the device) to facilitate handling by the user, while a proximal end (e.g. of the tubular member) may be attached to a benchtop or cart-based machine, for example, or a mounted or fixed machine. In these variations, the grip may or may not contain any other components of the tissue removal device, such as a motor, while the machinery at the proximal end (e.g. of the tubular member) may contain one or more other components, such as a suction system or various radiofrequency ablation components, for example. In some variations, the housing may have a length of about 1 cm to about 12 cm or more, sometimes about 2 cm to about 8 cm, and other times about 3 cm to about 5 cm. The average diameter of the housing (or other transverse dimension to the longitudinal axis of the housing) may be about 1 cm to about 6 cm or more, sometimes about 2 cm to about 3 cm, and other times about 1.5 cm to about 2.5 cm. The housing may further comprise one or more ridges, recesses or sections of textured or frictional surfaces, including but not limited to styrenic block copolymers or other polymer surfaces.

Tissue removal assembly 1108 further comprises a jaw member 1110 comprising an upper jaw portion 1112 and a lower jaw portion 1114. The jaw member 1110 is coupled to tubular member 1106, with the lower jaw portion 1114 essentially extending from the tubular member 1106. Lower jaw portion 1114 and tubular member 1106 are fixedly coupled to collection chamber 1105. Additionally, the upper jaw portion 1112 is coupled to an actuating elongated member 1116 that, in turn, is coupled to an actuation knob 1118, which is also coupled to tubular member 1106. In some cases, elongated member 1116 may be tubular or semitubular. An impeller 1120, which may be coupled to a rotating drive shaft and/or an auger (both not shown), is at least partially disposed within lower jaw portion 1114, in a sheath tip 1122. In other variations, sheath edges may alternatively or additionally be cut into the internal surfaces of lower jaw portion 1114 and/or upper jaw portion 1112. In cases in which such sheath edges are used as an alternative to an actual sheath, the use of the sheath edges may, for example, advantageously save on wall thickness stack up.

In use, an operator may actuate trigger 1109 to plunge the auger and impeller back and forth. The actuation knob 1118 may be actuated (e.g. prior to, during and/or after actuation of the trigger 1109) to open and close the upper jaw portion 1112 as desired. In other words, the actuation knob 1118 may be turned or otherwise manipulated to linearly move the elongated member 1116 and thereby open and close upper jaw portion 1112. In some cases, one hand may be used to actuate the trigger 1109, and the opposite hand may be used to actuate the actuation knob 1118. In some cases, the trigger 1109 may be actuated (e.g. pulled on) to plunge the auger/impeller, and the jaw portions 1112 and 1114 may simultaneously be closed toward each other. Actuation of the trigger 1109 may occur by pressing the trigger 1109, thereby causing trigger extension 1124, which is pinned only to actuating elongated member 1116, to slide forward and close upper and lower jaw portions 1112 and 1114 toward each other. In some cases, a spring or other resilient element (not shown) may fit over a driveshaft coupler 1126 between a distal bearing 1128 of the tissue removal device 1100 (FIG. 11C) and trigger 1109, to spring jaw portions 1112 and 1114 open. In a tissue removal procedure, jaw portions 1112 and 1114 may be actuated to cut and/or remove tissue, and impeller 1120 may be actuated to break the tissue apart. In some cases, the tissue removal device 1100 may further comprise an auger or other tissue transport assembly (not shown) that may be used to proximally transport the collected tissue.

Some variations of tissue removal devices described herein may also be capable of aspirating tissue. For example, a tissue removal device may comprise a conduit which may be used to connect the tissue removal device to an aspiration or suction source. An aspiration or suction source may be used, for example, to transport fluid or material through a lumen or conduit of a tubular member of the tissue removal device. In certain variations, one or more separate ports may be provided for infusing or injecting substances into a target site using the tissue removal device. In other variations, the above-described conduit may be used for both withdrawal and infusion of materials and/or fluids, or for infusion only. Depending upon the configuration of the tissue removal device, withdrawal and/or infusion may occur at the distal end of the device, and/or through one or more openings of the tissue removal assembly of the device. In some variations, a port may be used to insert a coagulation catheter, an ablation catheter, or another energy delivery device to the target site.

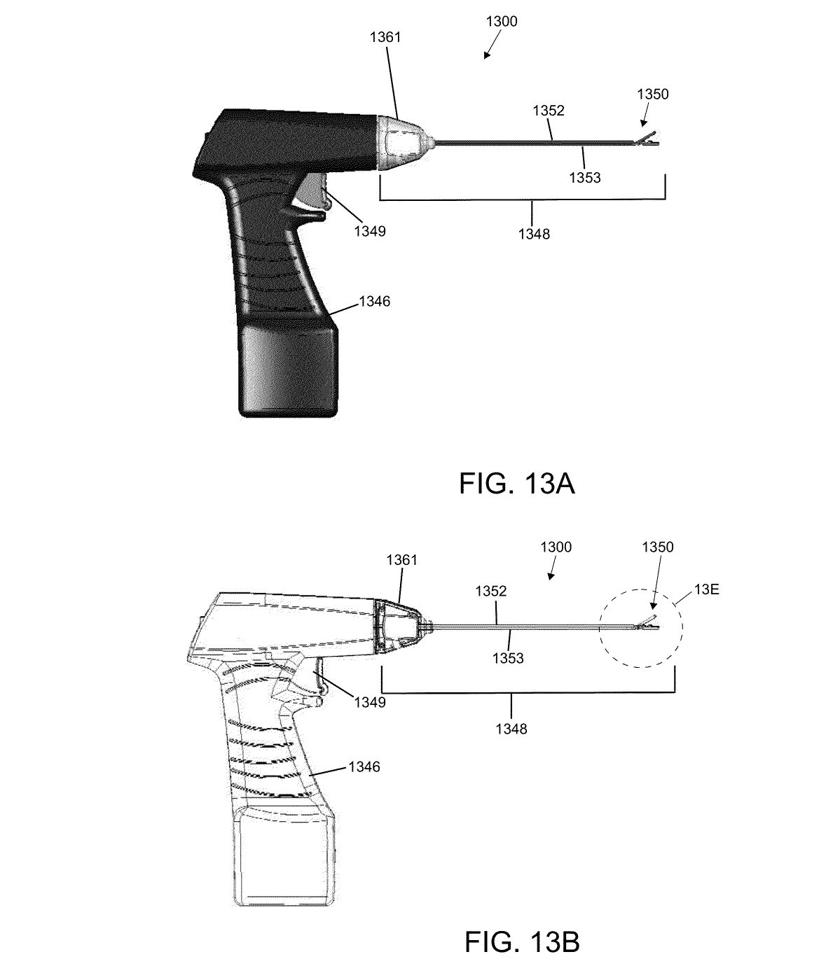

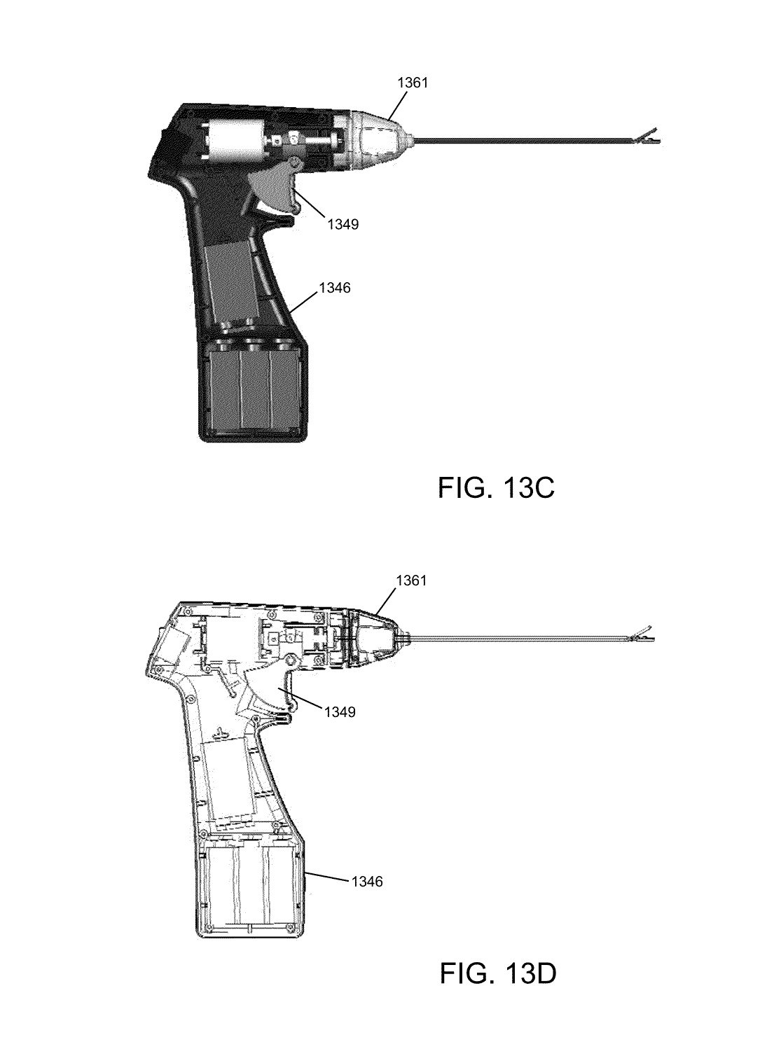

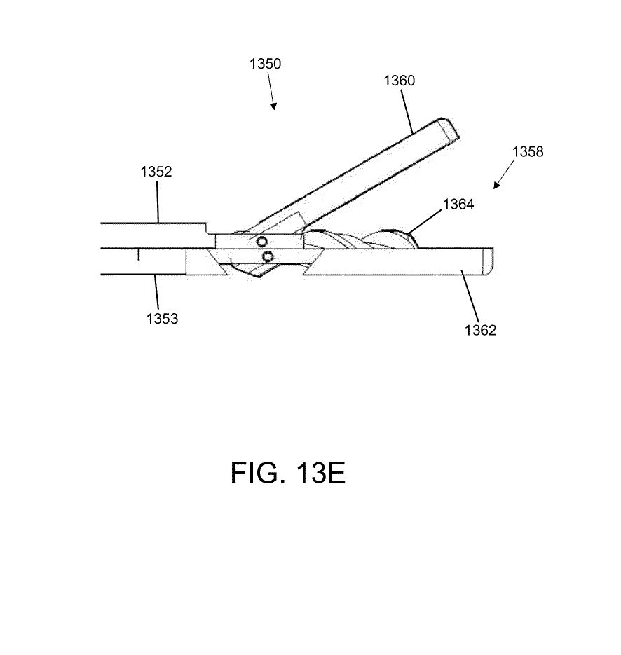

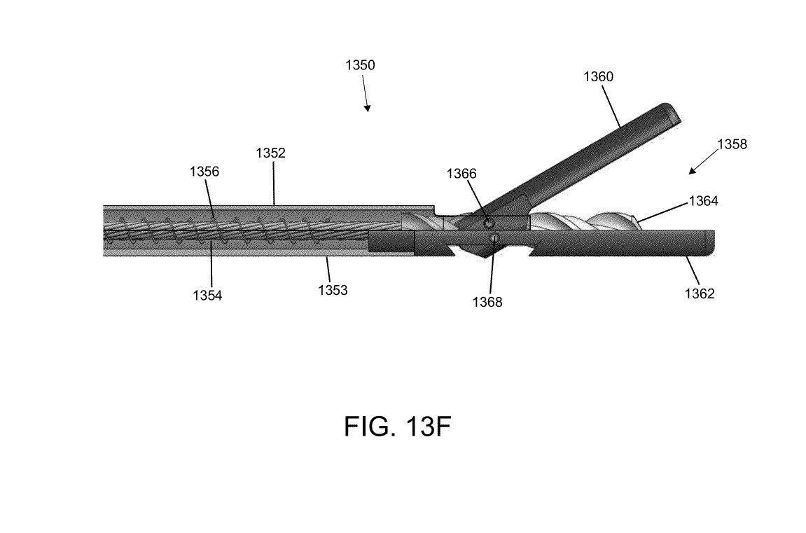

Other variations of tissue removal devices including tissue removal assemblies comprising jaws and/or jaw-like features may be employed in tissue removal procedures. For example, FIGS. 13A-13F depict a tissue removal device 1300 comprising a housing 1346 and a tissue removal mechanism 1348 coupled to the housing and comprising a collection chamber 1361 and a tissue removal assembly 1350. A trigger 1349 that is coupled to housing 1346 may be used to actuate tissue removal mechanism 1348. Housing 1346 may, for example, comprise one or more of the same components (e.g. in the same configuration) as other tissue removal devices housings described herein.

Tissue removal assembly 1350 includes an upper semitubular member 1352, a lower semitubular member 1353, a rotatable shaft 1354 disposed within the upper and lower semitubular members, an auger 1356 disposed around the rotatable shaft, a jaw member 1358 comprising upper and lower jaw portions 1360 and 1362, respectively (as shown, in the form of half-tubes), and an impeller 1364 coupled to rotatable shaft 1354 and disposed within lower jaw portion 1362. Upper jaw portion 1360 is coupled to upper semitubular member 1352, and lower jaw portion 1362 is coupled to lower semitubular member 1353. Referring specifically to FIG. 13F, hinges 1366 and 1368 (as shown, in the form of hinge pins) couple the upper and lower jaw portions to each other, and allow the upper jaw portion to be opened relative to the lower jaw portion.

Hinges 1366 and 1368 may be located at any suitable position and in some cases may be set to control the maximum angle (e.g. about 30°) at which the upper jaw portion 1360 opens with respect to the lower jaw portion 1362. Setting the hinge pin holes further apart on the upper and lower jaw portions 1360 and 1362 may allow the jaw member 1358 to open at a greater angle. For example, jaw member 1358 may open at an angle of more than 30 degrees, such as 45 degrees, which may desirably allow for a greater volume of tissue to be manipulated. While hinge pins are used here, other variations of tissue removal devices may employ different hinging components or mechanisms.

In use, jaw member 1358 may fill with grabbed tissue and then impeller 1364 may be plunged distally to break up the tissue. When jaw member 1358 is open, impeller 1364 may be proximal to the jaw portions 1360 and 1362, to provide space within jaw member 1358 for tissue grabbing and collection. In some cases, jaw member 1358 may comprise one or more features, such as sharp, chamfered and/or serrated edges, that may be used to help cut the target tissue prior to grabbing the tissue. As upper and lower jaw portions 1360 and 1362 are actuated to close, impeller 1364 may (e.g. simultaneously) plunge distally to drive through the collected tissue. The timing of the jaw action and the impeller action may be coordinated, for example, by adjusting a trigger extension of the tissue removal device (e.g. like trigger extension 1124). As an example, the location at which the holes of the trigger extension mate to upper semitubular member 1352 within housing 1346 and/or the ramp angle of trigger 1349. Once tissue has been collected and broken up, auger 1356 may facilitate the transporting of the tissue to collection chamber 1361.

When hand tools such as pituitaries are used in a tissue removal procedure, a significant grabbing force may be employed to pull or yank away a tissue piece. Here, serrations, internal chamfers and/or other suitable features may be added to upper and/or lower jaw portions 1360 and 1362 (e.g. to their edges), thereby allowing the upper and lower jaw portions to effect an initial cutting action to separate the tissue prior to the secondary shearing by impeller 1364 and the sheath, if any.

In cases in which a sheath is used around an impeller (e.g. as described above), the sheath tip geometry may be selected to provide a good cutting edge. In some cases, the fluted regions on impeller 1364 may be located at approximately a 40 degree angle and the shearing edge length of the tip of a sheath tip (not shown) surrounding impeller 1364 may be maximized for contact with the flute edge. In certain variations, the shearing edge may be at an opposing angle relative to the flute angle, but may be kept to the horizontal axis.

The various tissue removal devices disclosed herein may be used to perform a discectomy or nucleotomy, but may also be used to perform any of a variety of tissue removal procedures in the spine and outside of the spine. Examples of procedures that may be used to access the spine are disclosed in U.S. Pat. No. 7,108,705, U.S. Pat. No. 4,573,448, U.S. Pat. No. 6,217,5009, and U.S. Pat. No. 7,273,468, which are hereby incorporated by reference in their entirety.

The tissue removal devices may be used in minimally invasive procedures as well as open surgical procedures or limited access procedures. These procedures may include but are not limited to interlaminar, translaminar and intralaminar access procedures. In one particular embodiment, a patient may be placed into a prone position with a pillow or other structure below the abdomen to limit lumbar lordosis. The patient may be prepped and draped in the usual sterile fashion and anesthesia may be achieved using general, regional or local anesthesia. Under fluoroscopic guidance, a sharp tipped guidewire, or a needle with a guidewire, may be inserted into the paravertebral space or epidural space from a posterior or postero-lateral location of the patient's back at a location in the range of about 2 inches to about 6 inches lateral to the midline. In some instances, guidewire insertion may be facilitated by inserting a needle into the tissue first. In alternate variations, an anterior procedure through the abdominal cavity or anterior neck region may be performed. Once access to the target location is confirmed, a dilator may be used with the guidewire to enlarge the insertion pathway. Then, an introducer or cannula may be inserted over the guidewire, followed by subsequent guidewire removal and insertion of an endoscope into the introducer or cannula. Alternatively, an endoscope may be inserted over the guidewire. The endoscope may be manipulated or steered to directly visualize and identify the relevant structures such as the disc, the nerve or other adjacent structures and site(s) of tissue removal. In some variations where the patient is under local or regional anesthesia, a suspected nerve impingement may be confirmed by contacting or manipulating the suspected nerve with the endoscope, or other device inserted through the endoscope, and assessing the patient's response or symptoms. One variation of an endoscope that may be used is described in U.S. patent application Ser. No. 12/199,706, which is hereby incorporated by reference in its entirety.

Once the target region has been evaluated, a tissue removal device may be inserted through the spinal access device or endoscope and to pierce through the annular wall of a herniated disc. Once inserted, the tissue removal device may be manipulated and actuated to remove the target tissue. In some variations, the tissue removal device may be actuated for a duration in the range of about 5 seconds to about 90 seconds or more, sometimes about 15 seconds to about 60 seconds, and other times about 30 seconds to about 60 seconds.

In certain variations, any collected material may be suctioned through the device and then the effect of the tissue removal may be re-evaluated by the endoscope or other visualization mechanisms. In some variations, a liquid or lubricant may be injected or infused into the treatment site. In some examples, the liquid or lubricant may be useful to facilitate removal of the collected material, including but not limited to vertebral discs that may be desiccated. In other examples, the liquid or lubricant may be injected or infused before or during the actuation of the tissue removal device. In some examples, the liquid or lubricant may comprise a contrast agent that may facilitate viewing of the tissue site on fluoroscopy, x-ray, CT, MRI, ultrasound or other imaging modalities. The contrast agent may be used at any time or at multiple times during the procedure, including but not limited to confirmation of guidewire or tissue removal device placement, and also to verify the volume and/or location of tissue removal.

In some specific variations, actuation of the tissue removal device may be stopped to verify that the annulus of the vertebral disc or the cortical bone of the vertebral body has not been compromised. Also, in some examples, contrast agent may be injected and imaged after device actuation to assess proper operation of the device, including but not limited to tissue pulverization and aspiration mechanisms.

During actuation, the tissue removal device may be held in place or may be moved around the treatment site. Suction or aspiration may be applied during these motions to assess the amount of tissue being removed.

The actuation of the tissue removal device may be repeated as desired to remove disc material. In some embodiments, the tissue removal device may be withdrawn from the disc and reinserted directly into or against the extruded disc material and actuated. Once the tissue removal is completed, the tissue removal device may be withdrawn. The puncture site in the annular wall may have a cross-sectional area of less than about 0.003 inch² or less, sometimes about 0.0016 inch² or less, and other times about 0.001 inch² or less, and thus may self-seal without requiring treatment of the puncture location with an adhesive, a suture or coagulation probe. The body location may be rechecked with the endoscope or spinal access device to verify that no bleeding or compromise of the integrity of the disc or spinal nerves has occurred, and then the endoscope or spinal access device may be removed from the body and the skin access site may be bandaged.

While various tissue removal devices may be used to remove larger volumes of tissue, in other variations, a tissue removal device may be used to perform focal debulking of tissue. For example, by utilizing the small profile and/or the steerable features of certain variations of the tissue removal device, the tissue removal device may be more accurately positioned or navigated to a specific target site in a body structure. In some instances, the removal of lower volumes of tissue at a specific target location may be used to achieve a desired result, in comparison to the removal of a larger volume of tissue from a general target location. By removing less disc tissue to reduce a herniation, for example, a larger amount of non-pathologic disc tissue and structural integrity of the disc may be preserved. In some instances, relatively greater preservation of the disc tissue may slow the rate of further disc degeneration and reherniation compared to lesser degrees of tissue preservation.

In one example, a herniated disc may be accessed and visualized endoscopically. A steerable tissue removal device may be inserted into the disc and steered toward the region of herniation, rather than to the center of the disc, for example.

The procedures described herein may target vertebral tissue in different locations, and as such, access sites and pathways may vary accordingly. The tissue removal devices described above may be used with one or more access devices which may help direct the tissue removal device to the target tissue site. An access device, such as a cannula, may be positioned with different angles of entry depending on the location of the targeted vertebral tissue. The range of suitable entry angles may be at least partially constrained by the location of spinal structures with respect to the skin surface. For example, a straight cannula may be positioned within the range of suitable entry angles to create a linear access pathway that extends from an access site on the skin surface to a targeted region of spinal tissue that is co-linear with access site. A curved cannula may be used to create a curved pathway to access tissue that may not be co-linear with an access site within a suitable entry angle range. While a curved pathway may provide increased accessibility to vertebral tissue, a practitioner may need to undergo additional training and practice to avoid disrupting sensitive anatomical structures along a curved pathway. Some variations of access devices may comprise a bendable flexible curvable cannula, which may have a straight configuration and a curved configuration. The cannula may be used in the straight configuration to create a substantially linear access pathway from the access site on the skin surface to the vicinity of the target vertebral tissue. Once the initial access pathway is created, the cannula may be used in the curved configuration to contact the target tissue.

In some variations, the curvature of a cannula may be determined in part by the curvature of a stylet inserted therethrough. For example, inserting a stylet with one or more curves into a bendable flexible cannula may cause the cannula to have corresponding curves. In some variations, a bendable cannula may have one or more pre-formed curves that may be straightened by inserting a straight stylet therethrough. Alternatively, a bendable cannula that is substantially straight may be curved by inserting a curved stylet therethrough. The insertion of various stylets through a bendable cannula may allow a practitioner to access spinal tissue at different locations via one access site on the skin. This may reduce the need for withdrawing the cannula from the body and re-entering the body via an additional access site to access a different tissue region. For example, the cannula and the stylet may each have one or more corresponding curves such that when the stylet is inserted through the cannula, the corresponding curves may be aligned. This may act to stiffen or reinforce the curvature of the cannula so that it may be more easily moved from a first tissue location to a second tissue location. For example, a procedure performed on one tissue location in the disc annulus may be repeated at another tissue location without removing the curved cannula from the disc annulus. While at the first tissue location, a curved or straight stylet may be reintroduced into the cannula, which may facilitate adjustment and positioning of the cannula to a second tissue location. Insertion of a straight stylet may straighten the curved portion of the cannula and allow the cannula-stylet assembly to be advanced to a target site that is relatively further away from the site that has been treated. In other embodiments where relatively insignificant cannula repositioning is involved, a curved stylet may be used to acquire access to a second target site within the disc. A straightened and/or stiffened cannula-stylet assembly may offer enhanced responsiveness and maneuverability and therefore facilitate the maneuvering of the cannula within the discal area, and may facilitate safe removal of the devices from a patient.

The length of a stylet may be greater than, or substantially equal to the length of a corresponding cannula. For example, the distal portion of a stylet inserted into a cannula may extend or protrude from the distal portion of the cannula, and/or may be flush with the distal portion of the cannula, and/or may even be withdrawn into the cannula, as desirable. Similarly, the tissue removal assembly of a tissue removal device may be extended from and/or withdrawn into the distal portion of the cannula. The relative longitudinal position between a cannula and stylet, and/or cannula and a travel limiter of a tissue removal device may be adjusted and/or locked. In some variations, the orientation of one or more curves in a cannula and a stylet with respect to each other may be adjusted by rotating the stylet, and may optionally be locked once the desired orientation is obtained. The cannula and stylet may each comprise complementary proximal connectors, which may be used to couple them together, such that they may be advanced and navigated together. Optionally, the proximal connectors may rotatably and/or longitudinally lock the cannula and stylet with respect to each other.

Some variations of a cannula and/or stylet may have an orientation indicator, which may help a practitioner to identify the orientation of the one or more curves of the devices, or the orientation of one or more sharpened edges of a stylet, after they have been inserted into the body of a patient. For example, the orientation of a distal curve of a cannula with respect to the longitudinal axis of the cannula shaft may be evident by observing the configuration of the orientation indicator. Orientation indicators may also help a practitioner align the curvature of a stylet to correspond with the curvature of the cannula that it is inserted through. In this way, the practitioner may proximally adjust the bend orientation of the stylet, thereby allowing the stylet to pass through the cannula bend with ease. The shape of the orientation indicator may convey the orientation of the one or more curves of the cannula and/or style to the practitioner. For example, the orientation indicator may have a shape with one or more tapered regions, where the plane of a taper is indicative of the plane of a distal curve. In some variations, orientation indicators may have multiple apices that are aligned with multiple curves in multiple planes, which may help the practitioner position and orient the distal portion of the tissue removal device as desired. The orientation indicator may be attached to the cannula and/or stylet by soldering, welding, adhesive bonding (e.g., 3311 UV adhesive that may be UV cured), snap fit, or other appropriate methods. In some variations, the orientation indicator may be attached or integrally formed with a proximal connector of the cannula and/or stylet. This may provide a mechanism for the cannula and stylet to be coupled together in a particular orientation.

Cannulas and stylets may each have proximal connectors that couple them to each other. The proximal connector of a cannula may also be used to couple it with a tissue removal device, e.g., a collector port and/or travel limiter. Connectors may be any standardized connector (e.g., any luer-type connectors, screw-type connectors, taper ground joints, etc.), or may be a proprietary connector. In some variations, a cannula may have a male-type connector that is configured to connect with a stylet or tissue removal device with a female-type connector. Engagement of the proximal connectors of cannula, stylets, and/or tissue removal devices may prevent relative movement between the devices. In some variations, when a stylet is connected to a cannula, the stylet may not be able to move longitudinally within the cannula, but may be axially rotated within the cannula. This may allow a practitioner to adjust the alignment between the cannula and stylet during the insertion of the cannula and stylet into the body. Alternatively or additionally, engagement of the proximal connectors between a cannula and stylet, or a cannula and a travel limiter of a tissue removal device may prevent relative longitudinal and axial motion between the devices. Locking the orientation and position between the cannula and stylet (and/or cannula and travel limiter) may help prevent inadvertent device misalignment or movement during a procedure. Travel limiters are disclosed, for example, in U.S. Patent Application Ser. No. 61/425,226, which is incorporated herein by reference in its entirety.

In some examples, the distal region of the cannula and/or stylet may comprise a radio-opaque structure (e.g. rings or bands) to facilitate confirmation of its position using radiographic imaging. In other examples a separate radiographic marker instrument may be used to confirm and evaluate the cannula placement.

In some variations, a bendable flexible curved cannula may be used in association with either a straight stylet or a curved stylet to obtain curved access to a spinal area. A curved access pathway not only offers a larger tissue removal zone at one target site, but it may also provide flexible access to multiple target sites in one or more herniated discs. A curved or non-linear access pathway that may be provided by a bendable flexible curved cannula may be shorter than a straight access pathway, and may be less disruptive to surround tissue structures. It may also provide better orientation towards the middle of a disc, as compared with a straight access pathway.

The bending range of the curved cannula may be in the range of from about 10 degrees to about 80 degrees, sometimes from about 20 degrees to about 70 degrees, and other times from about 30 degrees to about 60 degrees, and still other times from about 40 degrees to about 50 degrees. The curved distal portion 2914 may comprise a radius of curvature of about 0.5 centimeter to about 30 centimeters; sometimes about 1 centimeter to about 20 centimeters, sometimes about 5 centimeters to about 15 centimeters and other times about 8 centimeters to about 10 centimeters. When the curved distal portion is straightened, the curved cannula may comprise a length of about 4 inches to about 12 inches or more, sometimes about 5 inches to about 10 inches, and other times about 6 inches to about 9 inches.

Prior to inserting the tissue removal device into the cannula, approximately 0.5 cc of saline may be injected into the disc through the cannula. Under image guidance, the tissue removal device may be inserted through the cannula until the target site has been reached. Using image guidance, the practitioner may advance the tip of the tissue removal device to the full plunge depth, and confirm that the tip is in a safe location. The tissue removal device may then be actuated. The placement of the device in the course of tissue removal may be intermittently confirmed by fluoroscopy or another appropriate imaging modality. The tissue removal device may be used until sufficient tissue material has been removed, and/or the collector is full. In some variations, a negative pressure source may be coupled to the collector which may help expedite tissue removal. The markings on the collector indicate the quantity of tissue removed. The tissue removal device may be turned on and used continuously for about 0.5 second to about 6.0 minutes, e.g., 2.0 minutes.

Once a sufficient quantity of tissue material has been removed, the tissue removal device may be turned off. The above steps may be repeated until the desired quantity of tissue has been removed. If additional treatment is required within the disc, the straight or curved stylet may be reinserted into the cannula, and the cannula may be repositioned. In some procedures, it may be desirable to limit the total run-time of the tissue removal device to about 6.0 minutes or less. The straight stylet may be inserted into the cannula and fixedly attached at the proximal hub. Then, the cannula-straight stylet assembly may be withdrawn from the access site. In some variations, the battery of the tissue removal device may be removed and disposed according to local regulations.

The cannula, stylet, and tissue removal devices described above may be used to perform a discectomy. The devices may be used in a minimally invasive procedure, or an open surgery procedure. The cannula-stylet assembly may be used to form a passageway or a working channel through the tissue about a target site in the spinal region. For example, to perform a discectomy procedure, the patient may be prepped and draped in the usual sterile fashion and in a lateral decubitis or prone position. General, regional or local anesthesia may be achieved. A straight stylet with a sharp distal tip may be inserted into the lumen of a straight cannula. The assembly may then be percutaneously inserted through a posterior or posterolateral entry point on the back of the patient. The cannula-stylet assembly may be further inserted into the epidural space or into the paravertebral space, depending on the assembly's point of entry. Alternatively, the assembly may be used to penetrate the disc annulus directly from a point of entry further away from the midline of the patient's back. In some embodiments, the assembly may be introduced on the ipsilateral side from which the nerve impingement has been identified and at an angle of about 25 degrees to about 45 degrees to the patient's back. In other procedures, a contralateral approach and/or a different angle may be used. In alternative embodiments, an anterior procedure through the abdominal cavity of the anterior neck region may be performed.

The cannula-stylet assembly may be advanced together to a target tissue site, as described above. During the insertion of the assembly, the stylet may be independently rotatable such that the operator may adjust the orientation of the optional beveled edge of the stylet in order to form a passageway through the surrounding tissue, bones or other anatomic structures. The insertion of the cannula-stylet assembly may be performed under the guidance of external imaging and/or visualization techniques.

Fluoroscopy and/or CT scan may be used before, during and/or after the procedure to assess the patient's anatomy, the position of the instruments, the structural changes after tissue removal, and/or to verify the integrity of the disc. In some variations, a small amount of radiopaque contrast agent may be injected into the disc space to enhance visualization. Such injection may be performed by the tissue removal device through an infusion or irrigation channel, or through the aspiration port. In other variations, the cannula may comprise an infusion or irrigation lumen to introduce the contrast agents. In some variations, the tissue removing procedure may be assessed by the quantity and/or color of the tissue removed through an optically transparent chamber, or collection chamber. Upon completion of the procedure, the tissue removal device may be proximally withdrawn, followed by withdrawal of the cannula.

Devices described herein may be used with one or more visualization systems, such as one or more endoscopic visualization systems, as appropriate.

It is to be understood that this invention is not limited to particular exemplary variations described, as such may, of course, vary. It is also to be understood that the terminology used herein is for the purpose of describing particular variations only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a blade” includes a plurality of such blades and reference to “the energy source” includes reference to one or more sources of energy and equivalents thereof known to those skilled in the art, and so forth.

The publications discussed herein are provided solely for their disclosure. Nothing herein is to be construed as an admission that the present invention is not entitled to antedate such publication by virtue of prior invention. Further, the dates of publication provided, if any, may be different from the actual publication dates which may need to be independently confirmed.

What is claimed is:

1. A tissue removal device comprising:
a handheld housing;
a motor; and
a tissue removal mechanism coupled to the handheld housing, the tissue removal mechanism comprising:
a tissue collection chamber coupled to a distal portion of the handheld housing;
a jaw member comprising first and second jaw portions, wherein the first jaw portion is coupled to a first elongated member and the second jaw portion is coupled to a second elongated member configured to actuate the second jaw portion;
a rotatable shaft disposed within a lumen of the first elongated member;
a helical member disposed around at least a portion of the rotatable shaft; and
an impeller coupled to at least one of a distal end of the helical member and a distal end of the rotatable shaft,
wherein rotation of the rotatable shaft effects rotation of the helical member and the impeller.

2. The tissue remove device of claim 1, wherein one of the first and second jaw portions includes an edge configured to cut tissue.

3. The tissue removal device of claim 2, wherein the edge is a sharp edge.

4. The tissue removal device of claim 2, wherein the edge is a chamfered edge.

5. The tissue removal device of claim 2, wherein the edge is a serrated edge.

6. The tissue removal device of claim 2, wherein the edge includes one or more of a sharp edge, a chamfered edge, and a serrated edge.

7. The tissue removal device of claim 2, wherein the first jaw portion includes a first edge and the second jaw portion includes a second edge, each of the first and second edges including one or more of a sharp edge, a chamfered edge, and a serrated edge.

8. The tissue removal device of claim 2, wherein an inside surface of one of the first and second jaw portions included a groove, the groove defining a portion of the edge.

9. The tissue removal device of claim 1, wherein a portion of the rotatable shaft is positioned within one or the first and second jaw portions.

10. The Tissue removal device of claim 1, wherein the helical member includes a first cutting edge, and one of the first and second jaw portions includes a second cutting edge, the first cutting edge cooperating with the second cutting edge to cut a portion of tissue.

11. The tissue removal device of claim 1, wherein actuation of the second jaw portion is achieved through translation of the second elongated member.

12. The tissue removal device of claim 1, wherein the rotatable shaft is configured to translate with respect to the jaw member.

13. The tissue removal device of claim 1, wherein the rotatable shaft is slidably disposed within the first jaw portion.

14. The tissue removal device of claim 1, wherein the first jaw portion is coupled to the second jaw portion through a hinge.

15. A tissue removal device comprising:
a handheld housing;
a motor; and
a tissue removal mechanism coupled to the handheld housing, the tissue removal mechanism comprising:
a tissue collection chamber coupled to a distal portion of the handheld housing;
a tubular member;
a rotatable elongated member disposed within the tubular member; and
a tissue removal assembly comprising a helical member disposed around at least a portion of the rotatable elongated member, an impeller coupled to a distal portion of at least one of the rotatable elongated member and the helical member, and a distal opening configured to receive and funnel tissue into a narrowed region and toward the impeller, wherein the opening is bounded by a hood and an inner sheath within the hood, and wherein rotation of the rotatable shaft effects rotation of the helical member and the impeller.

16. The tissue removal device of claim 15, wherein the hood includes a backward-facing edge configured to remove a portion of tissue.

17. The tissue removal device of claim 16, wherein the backward-facing edge cooperates with the impeller to remove the portion of tissue.

18. The tissue removal device of claim 15, wherein the hood includes a groove having an edge configured to cut a portion of tissue.

19. The tissue removal device of claim 18, wherein the edge is a sharpened edge.

20. The tissue removal device of claim 18, wherein the edge is a serrated edge.

21. The tissue removal device of claim 18, wherein the edge includes first and second portions, the first portion of the edge including a sharpened edge and the second portion of the edge including a serrated edge.

22. The tissue removal device of claim 18, wherein the hood includes a protrusion for disrupting tissue.

23. The tissue removal device of claim 18, wherein the hood includes a recess for disrupting tissue.
